(19) United States
(12) Patent Application Publication
(10) Pub. No.: US 2009/0270862 A1
(43) Pub. Date: Oct. 29, 2009

(54) MEDICAL DEVICE WITH ONE-WAY ROTARY DRIVE MECHANISM

(76) Inventor: Greg Arcenio, Redwood City, CA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/109,565

(22) Filed: Apr. 25, 2008

(51) Int. Cl. A61B 17/56; A61B 17/32

(52) U.S. Cl. 606/79

(57) ABSTRACT

Apparatuses and methods for accessing and disrupting a tissue are disclosed herein. In one embodiment, a method includes inserting a distal end portion of an elongate member into a biological body. After inserting the elongate member, an actuator is manually actuated to produce translational motion of a drive element. The translational motion is converted into rotational movement of the distal end portion of the elongate member. In one embodiment, an apparatus includes an elongate member having a tissue interaction member at a distal end portion that is configured to be inserted within a biological body. A conversion mechanism is coupled to the elongate member and includes a drive element. An actuator is coupled to the conversion mechanism and is configured to cause translational motion of the drive element. The conversion mechanism is configured to convert the translational motion of the drive element into rotational motion of the elongate member.

### CROSS-REFERENCE TO RELATED APPLICATIONS

This application is related to U.S. patent application entitled “Steerable Medical Device For Tissue Disruption,” Attorney Docket No. KYPH-041/01US 305363-2258, and U.S. patent application entitled “Medical Device For Tissue Disruption With Serrated Expandable Portion,” Attorney Docket No. KYPH-041/02US 305363-2257, both filed on the same date as this application, the disclosures of which are hereby incorporated by reference in their entirety.

### BACKGROUND

The invention relates generally to medical devices and procedures, including, for example, a medical device for percutaneously accessing a biological body, and disrupting tissue within the biological body.

Known medical devices are configured to access percutaneously a vertebra, an intervertebral disc, or other area of a spine to perform a variety of different medical procedures. Some known medical devices are configured to remove tissue from within the interior of a vertebra or intervertebral disc. Other known medical devices are configured to provide cutting means to tear, disrupt and/or loosen tissue within a vertebra or intervertebral disc.

In some medical procedures, a medical device used for disrupting tissue can be difficult to maneuver with the biological body. For example, it may be desirable to manually rotate a device while disposed within a biological body. Such manual rotation, however, may be difficult for the physician to perform. For example, it may be difficult for a physician to repeatedly twist his/her arm to rotate the medical device within a biological body. In addition, in some medical procedures the device used to disrupt tissue may need to be repeatedly removed from the biological body and reinserted potentially damaging the integrity of the biological body.

Thus, a need exists for an apparatus and method for disrupting tissue, such as tissue within an intervertebral disc or vertebra, where the apparatus can be expanded and collapsed, and rotated and/or maneuvered within the intervertebral disc or vertebra without repeated insertion and removal of the apparatus.

### SUMMARY OF THE INVENTION

Devices and methods for accessing and disrupting a tissue are disclosed herein. In one embodiment, a method includes inserting a distal end portion of an elongate member into a biological body. After inserting the elongate member, an actuation mechanism is manually actuated to produce translational motion of a drive element. The translational motion is converted into rotational movement of the distal end portion of the elongate member. In one embodiment, an apparatus includes an elongate member having a tissue interaction member at a distal end portion that is configured to be inserted within a biological body. A conversion mechanism is coupled to the elongate member and includes a drive element. An actuator is coupled to the conversion mechanism and is configured to cause translational motion of the drive element. The conversion mechanism is configured to convert the translational motion of the drive element into rotational motion of the elongate member.

### BRIEF DESCRIPTION OF THE DRAWINGS

FIG. 1 is a schematic illustration of a medical device according to an embodiment of the invention.

FIG. 2 is a side view of a medical device according to an embodiment of the invention.

FIG. 3 is a side view of a portion of the medical device of FIG. 2 shown with a portion of the housing removed and in a reset position.

FIG. 4 is side perspective view of a portion of the medical device of FIG. 2 shown with a portion of the housing removed and in a reset position.

FIG. 5 is a perspective view of a portion of the medical device of FIG. 2 shown with a portion of the housing removed and in a reset position.

FIG. 6 is a side perspective view of a portion of the medical device of FIG. 2 shown with a portion of the housing removed and in an actuated position.

FIG. 7 is a cross-sectional side perspective view of a portion of the medical device of FIG. 2 shown in a reset position.

FIG. 8 is a side perspective view of an expandable member according to an embodiment of the invention shown in an expanded configuration.

FIG. 9 is a schematic illustration of serrations according to an embodiment of the invention.

FIG. 10 is a side view of the medical device of FIG. 2 and an embodiment of a sheath.

FIG. 11 is a side view of the medical device of FIG. 2, the sheath of FIG. 10, and an access cannula shown disposed within a portion of a cross-sectional view of an intervertebral disc and a portion of two adjacent vertebrae.

FIG. 12 is a side view of an expandable member according to another embodiment of the invention.

FIG. 13 is a distal end view of the expandable member of FIG. 12 shown without serrations.

FIG. 14 is a side view of a medical device according to another embodiment of the invention shown with a portion of the housing removed and in a reset position.

FIG. 15 is a side view of a portion of the medical device of FIG. 14 shown with a portion of the housing removed and in a reset position.

FIG. 16 is a side view of a portion of the medical device of FIG. 14 shown with a portion of the housing removed and in an actuated position.

FIG. 17 is a side view of a portion of the medical device of FIG. 14 shown with the expandable member in a partially collapsed configuration.

FIG. 18 is a side view of a portion of the medical device of FIG. 14 shown with the expandable member in an expanded configuration.

FIG. 19 is a side view of the medical device of FIG. 14 shown partially disposed within an access cannula and within a cross-sectional view of a vertebra.

FIG. 20 is a side perspective view of a medical device according to another embodiment of the invention.

FIG. 21 is a cross-sectional side perspective view of a portion of the medical device of FIG. 20 shown in a reset position.

FIG. 22 is a side perspective view of a medical device according to another embodiment of the invention with a portion of the housing removed.

FIG. 23 is a side perspective view of a portion of the medical device of FIG. 22 shown in a first configuration.

FIG. 24 is a cross-sectional view of the portion of the medical device of FIG. 23.

FIG. 25 is side perspective view of the portion of the medical device of FIG. 23 shown in a second configuration.

FIG. 26 is a cross-sectional view of the portion of the medical device of FIG. 25.

FIG. 27 is a side cross-sectional view of a portion of the medical device of FIG. 22 shown with the steering mechanism in a first position.

FIG. 28 is a side cross-sectional view of a portion of the medical device of FIG. 22 shown with the steering mechanism in a second position.

FIG. 29 is aside perspective view of a distal end portion of a portion of a medical device according to another embodiment of the invention.

FIGS. 30-32 are each a flowchart illustrating a method according to different embodiments of the invention.

### DETAILED DESCRIPTION

The devices and methods described herein are configured for deployment within an interior area of a patient's body, such as within a hard tissue area (e.g., bone structure) or soft tissue area of a patient (e.g., intervertebral disc). For example, the devices can be percutaneously inserted within a biological body of a patient. In some embodiments, a device described herein is used to disrupt, sever, and/or cut a portion of a tissue within a biological body, such as a vertebra or intervertebral disc. In some embodiments, the apparatus and methods form a cavity within the biological body. For example, a medical device can include an expandable member that can be expanded while disposed within an interior area of a patient's body and rotated or otherwise maneuvered such that a cutting portion associated with the expandable member cuts tissue within the interior area of the patient.

In some embodiments, a medical device as described herein can be used to cut, tear, disrupt or scrape biological material within a biological body to form a cavity to allow a user to more easily insert an inflation balloon tamp (IBT) and reduce the likelihood of ruptures to the balloon during inflation. The medical devices described herein can include an expandable member at a distal end portion of the medical device. The expandable member can include one or more arms. The arms can be elastically-deformable. For example, the arms can be formed with, for example, a nitinol material or superelastic nitinol material such that they can be shape-set into a biased expanded configuration. The arms of the expandable member can be actuated between a collapsed configuration for insertion into a body, and an expanded configuration for use in distracting, scraping, tearing, and/or performing other operations on biological material within a tissue or biological body. The arms in the expanded configuration can, for example, have unconstrained ends (i.e., the tips of the arms are not attached to anything) and/or can each have a flared shape as described in more detail below.

The arms can be actuated, for example, using a sheath coupled to the expandable member. For example, the expandable member can be disposable within a lumen of the sheath. The sheath can be actuated to move between a first position in which the arms of the expandable member are disposed within the lumen of the sheath, and a second position in which the arms are disposed outside of the lumen of the sheath. In alternative embodiments, the sheath can be stationary and the expandable member can be moved relative to the sheath. For example, the expandable member can be moved between a first position in which the arms of the expandable member are disposed within the lumen of the sheath and a second position in which the arms are disposed outside of the lumen of the sheath.

A size (e.g., length, width, depth) of the arms and the quantity of the arms can be varied for use in different anatomical bodies, and to accommodate the formation of different sized cavities. For example, the size and/or pitch of the arms can be varied; the number and location of the arms can also be varied. In some embodiments, a medical device can have arms only on one side of the medical device. The medical device and arms can thus be sized or tailored for use in different medical procedures, and in different areas of anatomy.

In some embodiments, a medical device includes a rotary mechanism configured to rotate the arms when disposed within a biological body. For example, a rotary mechanism can be configured to rotate an elongate member in one direction and prevent the elongate member from rotating in an opposite direction. In some embodiments, a medical device can include a steering mechanism to assist in maneuvering a distal end portion of the medical device within a biological body.

In one embodiment, a method includes inserting a distal end portion of an elongate member into a biological body. After inserting the elongate member, an actuation mechanism is manually actuated to produce translational motion of a drive element. The translational motion is converted into rotational movement of the distal end portion of the elongate member.

In another embodiment, a method includes inserting a distal end portion of a medical device into a biological body such that a cutting member disposed at a distal end of the medical device is at a first location within the biological body. A tissue is disrupted at the first location within the biological body. The distal end portion of the medical device is reconfigured from a first configuration in which the distal end portion of the medical device has a first curvature to a second configuration in which the distal end portion of the medical device has a second curvature different than the first curvature and the cutting member is at a second location within the biological body. A tissue is then disrupted at the second location within the biological body.

In another embodiment, an apparatus includes an elongate member. A distal end portion of the elongate member includes multiple elastically deformable arms that are configured to perform a medical procedure in a biological body. The elastically deformable arms collectively have an unconstrained expanded configuration. Each of the elastically deformable arms has a serrated edge portion. The distal end portion of the elongate member can be rotated while disposed within a biological body such that the serrated edge portions of the arms disrupt tissue within the biological body.

In another embodiment, an apparatus includes a first elongate member and a flexible member disposed at a distal end portion of the first elongate member. A second elongate member is coupled to the first elongate member and is movable between a constrained configuration in which the flexible member is in a substantially linear configuration and an unconstrained configuration in which the flexible member is in a curved configuration. The first elongate member and the second elongate member are collectively configured to be inserted into a biological body when the second elongate member is in the constrained configuration. The flexible member is movable to the curved configuration while disposed within the biological body.

It is noted that, as used in this written description and the appended claims, the singular forms “a,” “an” and “the” include plural referents unless the context clearly dictates otherwise. Thus, for example, the term “a lumen” is intended to mean a single lumen or a combination of lumens. Furthermore, the words “proximal” and “distal” refer to direction closer to and away from, respectively, an operator (e.g., surgeon, physician, nurse, technician, etc.) who would insert the medical device into the patient, with the tip-end (i.e., distal end) of the device inserted inside a patient's body. Thus, for example, the end inserted inside a patient's body would be the distal end of the medical device, while the end outside a patient's body would be the proximal end of the medical device.

The term “tissue” is used herein to mean an aggregation of similarly specialized cells that are united in the performance of a particular function. For example, a tissue can be a soft tissue area (e.g., a muscle), a hard tissue area (e.g., a bone structure), a vertebral body, an intervertebral disc, etc. The terms “body” and “biological body” are also referred to herein to have a similar meaning as the term tissue.

The term “cutting portion” is used here to mean a component of an apparatus having at least one cutting surface and being configured to, for example, cut, sever, disrupt, scrape, or tear tissue. The cutting portion can be, for example, a cutting surface disposed on an elongate body, such as a cutting surface (e.g., serrations) disposed on an edge of an expandable portion of an elongate body. The cutting portion can also be a separate component coupled to a medical device.

The term “sheath” is used here to mean a component of the apparatus having one or more passageways configured to receive a device or other component. For example, a sheath can be substantially tubular. A sheath can be a variety of different shapes and size, such as having a round, square, rectangular, triangular, elliptical, or octagonal inner and/or outer perimeter. The sheath can be, for example, a cannula.

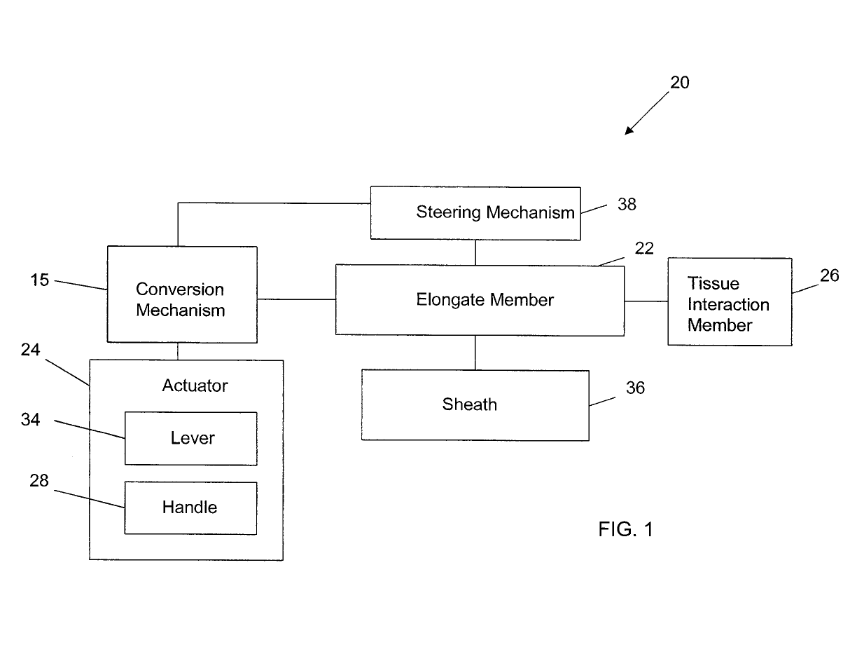

FIG. 1 is a schematic illustration of a medical device according to an embodiment of the invention. A medical device 20 can include an actuation mechanism 24, a conversion mechanism 15, an elongate member 22, and a tissue interaction member 26. The elongate member 22 can be coupled to the actuation mechanism 24. For example, a proximal end portion of the elongate member 22 can be coupled to the actuation mechanism 24. The conversion mechanism 24 can be disposed at least partially within a housing (not shown) and be coupled to the elongate member 22. The actuation mechanism 24 (also referred to herein as “actuator”) can include a lever 34 coupled to a handle 28. In some embodiments, the housing includes the handle 28.

In one embodiment, conversion mechanism 15 converts translational motion generated via actuation mechanism 24 (e.g., by the squeezing of the lever 34 toward the handle 28) into rotation of elongate member 22 and/or tissue interaction member 26. While rotating, the tissue interaction member 26 can perform a medical procedure in a biological body (e.g., disrupting tissue, extracting tissue, drilling in bone, inserting a bone screw, etc.). The conversion mechanism 15 allows a user of medical device 20 to generate rotational torque and motion to tissue interaction member 26 without having to repeatedly twist his/her arm, as would be required by conventional medical devices.

In some embodiments, the conversion mechanism 15 can include a threaded drive element (not shown in FIG. 1) configured to engage a threaded portion (not shown in FIG. 1) of elongate member 22 or a threaded portion of a separate component (not shown in FIG. 1) coupled to the elongate member 22. In some embodiments, the threaded portion of elongate member 22 can be, for example, a lead screw formed on or attached to elongate member 22. The threaded drive element can include a lead nut (not shown in FIG. 1) and a face gear (not shown in FIG. 1). In some embodiments, the drive element can alternatively include other components, such as for example, a drive nut, a gear, a pulley system, and/or a split nut. The conversion mechanism 15 can further include a return spring, a bronze bearing, and a pair of thrust bearings (not shown in FIG. 1). The medical device 20 can also include a rotation-limiting mechanism for allowing rotation of the elongate member 22 in only a single direction. The rotation-limiting mechanism can be, for example, a roller or rotary clutch (not shown), or other ratcheting mechanism as described in more detail below.

The threaded drive element and the threaded portion described above can have thread sizes that allow them to be freely threaded together. Conversion mechanism 15 and actuation mechanism 24 are configured to prevent rotation of the threaded drive element during proximal-to-distal and/or distal-to-proximal translational motion of threaded drive element (described in more detail below). For example, squeezing lever 34 and handle 28 together causes threaded drive element to be driven distally along the threaded portion. By preventing the threaded drive element from rotating during this translation along threaded portion, the threaded portion is forced to rotate, thereby rotating elongate member 22 (and tissue interaction member 26). In some embodiments, the conversion mechanism 15 is configured to rotate the elongate member 22 in a single direction. In other words, the conversion mechanism 15 will rotate the elongate member 22 in a first direction while preventing the elongate member 22 from rotating in a second opposite direction. The specific details of the function of the conversion mechanism 15 and actuation mechanism 24 are described in more detail below with reference to specific embodiments.

The tissue interaction member 26 is disposed at a distal end portion of the elongate member 22 and is configured to be inserted into a biological body, such as a vertebra or an intervertebral disc. The tissue interaction member can be coupled to the elongate member 22 or formed monolithically with the elongate member 22. The tissue interaction member 26 can be used to perform a medical procedure within the biological body, such as, for example, disrupting tissue, extracting tissue, drilling in bone, inserting a bone screw, etc. In some embodiments, the tissue interaction member 26 can be, for example, an expandable member. In some embodiments, the elongate member 22 is tubular (e.g., defines an inner lumen) and the tissue interaction member 26 (e.g., expandable member) is formed by laser cutting side walls of the elongate member 22 and shape-setting (e.g., heat-setting) the tissue interaction member 26 into an expanded configuration as described in more detail below.

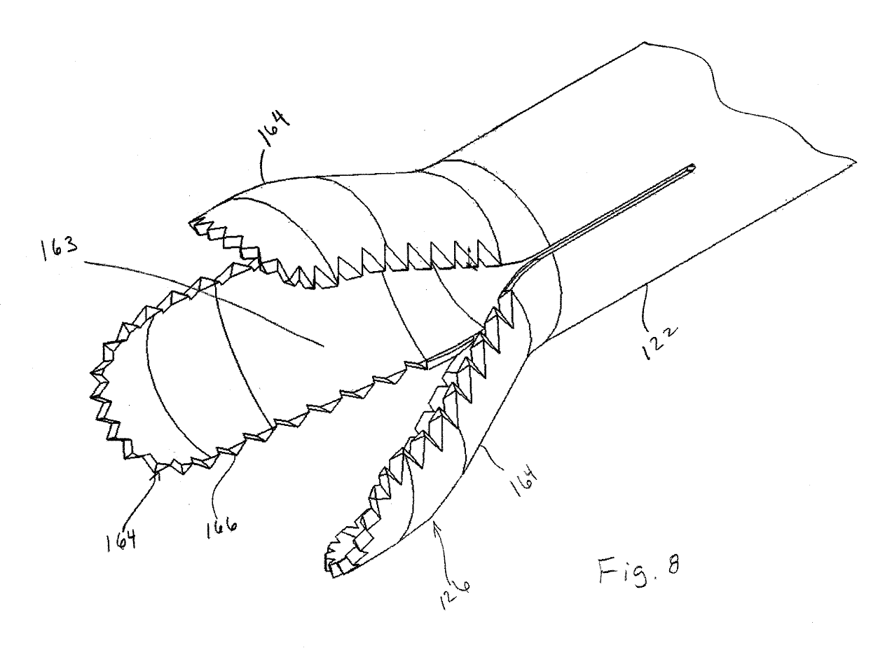

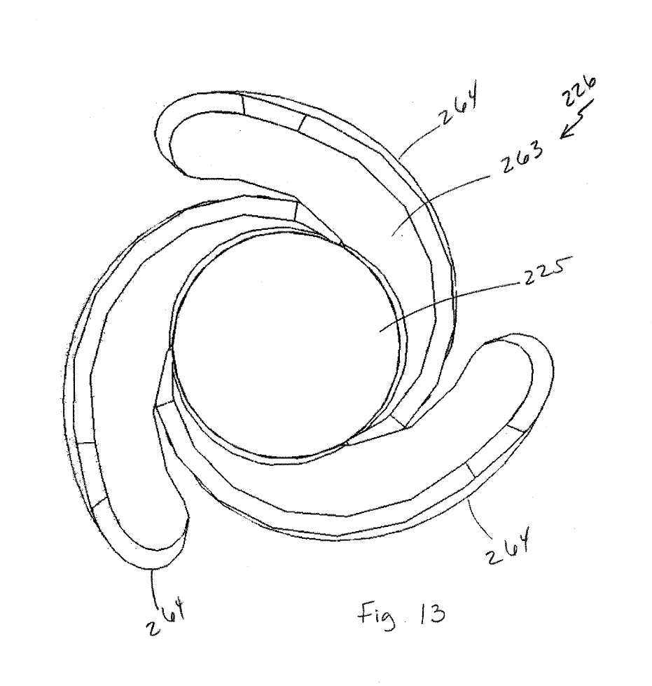

Such an expandable member can include multiple arms or tines that can be formed, for example, as described above, by laser cutting side walls of the elongate member 22. The multiple arms can be deformable. The multiple arms can extend or spiral outward from a tubular member such as the elongate member 22. The expandable member and/or the elongate member 22 can be formed with, for example, a shape-memory material (e.g., nitinol or superelastic nitinol) such that the arms of the expandable member can be biased into an expanded configuration by shape-setting the expandable member. In some embodiments, the arms have a flared shape when in the expanded configuration (e.g., an unrestrained, biased configuration) in that the arms collectively expand to an open configuration and the individual arms each have a curved or flared shape along its length. Such a flared shape is shown, for example, in FIG. 8, and discussed in greater detail below. In the expanded configuration, the arms can flare open to define an outer diameter that is, for example, 2 to 4 times larger than an outer diameter of the elongate member 22. In some embodiments the arms are in a spiral configuration as shown, for example, in FIG. 12, and discussed in greater detail below. In some embodiments, the arms can have a substantially linear or straight configuration when expanded (not shown).

The arms can also collectively be moved to a collapsed configuration by constraining the arms within, for example, a sheath 36. When the expandable member is disposed within the sheath 36 (described below), the arms will be collapsed. Thus, both the expandable member and the arms of the expandable member are referred to herein as having an expanded configuration and a collapsed configuration.

The arms can also include a cutting portion configured to cut or tear tissue. For example, the arms can include serrations along one or more edge of the arms. The serrations can cut or tear tissue within the biological body, for example, when the arms of the expandable member are moved within the biological body. In some embodiments, serrations are included only on a leading edge of the arm during rotation of the expandable member. The serrations can be formed, by laser cutting. For example, when the arms are formed by laser cutting side walls of the elongate body 22, as described above, the serrations can also be cut. The serrations can vary in size and quantity as described in more detail below.

In some embodiments, the elongate member 22 and tissue interaction member 26 (for example, a tissue interaction member having an expanded configuration as described above) can be movably disposed within the sheath 36, and the sheath 36 can be coupled to the actuation mechanism 24. In such an embodiment, the actuation mechanism 24 can move the sheath 36 proximally and distally relative the elongate member 22 such that the tissue interaction member 26 is moved from a position in which it is disposed within the sheath 36 and a position in which it is disposed outside of a distal end of the sheath 36. Thus, as the sheath 36 is moved, the tissue interaction member 26 is moved between its collapsed configuration (within the sheath 36) and expanded configuration (outside the sheath 36).

In some embodiments, a flexible member (not shown in FIG. 1) is coupled to a distal end portion of the elongate member 22. The flexible member is formed such that it can be moved between a first configuration in which it exhibits a first curvature (e.g., a substantially straight or linear configuration) and a second configuration in which it exhibits a second curvature (e.g., a substantially curved configuration). A steering mechanism 38 is used to move the flexible member between the two configurations and thus, steer or maneuver the elongate member 22 within a biological body as described in more detail below. A proximal end of the flexible member can be coupled to a distal end of the elongate member 22, or the flexible member and elongate member 22 can be formed as one component. The tissue interaction member 26 can be coupled to a distal end portion of the flexible member such that when the flexible member is maneuvered within a biological body, the tissue interaction member 26 will in turn be moved within the biological body.

In one embodiment, steering mechanism 38 can include a steering member (not shown in FIG. 1) disposed within a restraining element such as a steering sheath or tube (not shown in FIG. 1), which are both (i.e., steering member and steering sheath) partially disposed within a lumen of the elongate member 22. The steering member can be, for example, a steering rod. A proximal end portion of the steering mechanism 38 is coupled to the actuation mechanism 24, which can be configured to translate the sheath. The specific operation of the steering mechanism 38 is described in more detail below with reference to specific embodiments.

In one example use of the medical device 20, a distal end portion of the medical device 20 can be percutaneously inserted into a biological body, such as a vertebral body or an intervertebral disc. In this example, the tissue interaction member 26 is referred to as an expandable member as described above having collapsible arms. The distal end portion of the medical device is inserted into the biological body with the expandable member in a collapsed configuration (e.g., the arms collapsed within the sheath 36). In some embodiments, the medical device 20 is inserted through a separate cannula used to gain access to a tissue site. The expandable member can be moved to an expanded configuration while within the biological body and used to disrupt or tear tissue within the biological body. The medical device 20 can be actuated, for example using the lever 34 to actuate the actuation mechanism 24, and rotate the expandable member within the biological body (as described above). When the expandable member is rotated, the arms of the expandable member will scrape, disrupt or otherwise cut tissue within the biological body. The expandable member can then be moved to the collapsed configuration to allow the medical device 20 to be removed from the biological body.

The disrupted tissue within the biological body can then be removed using a separate medical device, such as a device configured to suction the disrupted tissue out of the biological body. In some embodiments the medical device 20 can be configured to be coupled to a suction source (not shown in FIG. 1). For example, a proximal end portion of the elongate member 22 can be coupled to a suction source, and disrupted tissue can be drawn or suctioned through a lumen of the elongate member 22. In some embodiments, a separate suction device can be inserted through the lumen of the elongate member 22 and used to suction disrupted tissue. Other procedures such as a procedure to inject bone cement into a cavity produced within the biological body by removal of disrupted tissue can also optionally be performed.

Having described above various general examples, several examples of specific embodiments are now described. These embodiments are only examples, and many other configurations and uses of the medical devices described herein are contemplated.

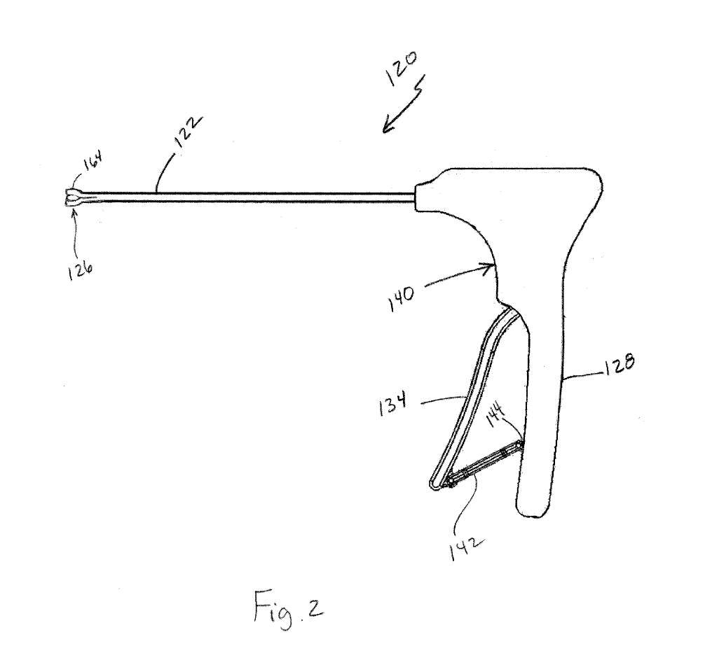

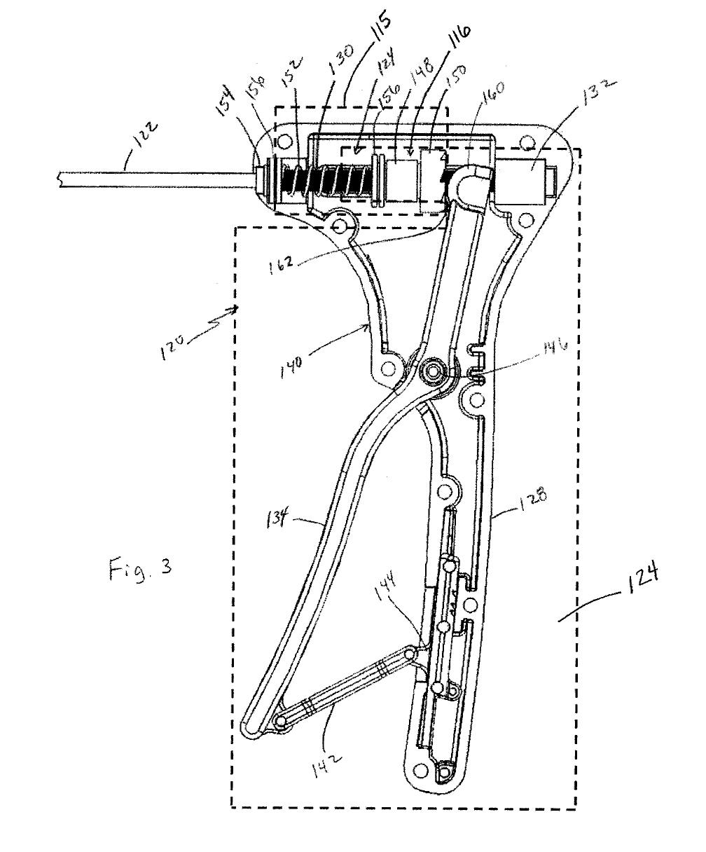

FIGS. 2-5 illustrate a medical device according to an embodiment of the invention. As shown in FIG. 2, a medical device 120 includes an elongate member 122 coupled to a housing 140 that includes a handle 128. At a distal end of the elongate body 122 is a tissue interaction member 126 (referred to herein as expandable member 126). As shown in FIG. 3, the elongate member 122 is coupled to a conversion mechanism 115, and the conversion mechanism 115 is coupled to an actuation mechanism 124. The actuation mechanism 124 includes a lever 134 that is coupled to the housing 140 via a pivot arm 142 and also at a pivot joint 146. The pivot arm 142 is also coupled to a slide member 144.

In this embodiment, the conversion mechanism 115 includes a threaded drive element 116, a rotation-limiting mechanism 132 (e.g., a roller clutch), a return spring 152, a bronze bearing 154, and a pair of thrust bearings 156. The threaded drive element 116 includes a drive nut 148 and a face gear 150. The elongate member 122 is coupled to a lead screw 130. The lead screw 130 has threads sized to matingly engage threads of the drive nut 148. As described above, the conversion mechanism 115 and the actuation mechanism 124 are configured to prevent rotation of the drive element 116 (e.g., the drive nut 148) during proximal-to-distal and/or distal-to-proximal translational motion of drive nut 148. For example, by squeezing lever 134 and handle 128 together, the drive nut 148 can be driven distally along threaded portion 130. By preventing drive nut 148 from rotating during this translation along threaded portion 130, threaded portion 130 is forced to rotate, thereby rotating elongate member 122 (and expandable member 126). The user can repeat the clutching motion of the lever 134 to produce repeated spurts of motion. The specific operation of the medical device 120 (and the various components of the medical device 120) is described in more detail below.

In this embodiment, the lead screw 130 is coupled to the elongate member 122, but as described above the lead screw 130 can alternatively be formed monolithically with the elongate member 122. The lead screw 130 can have, for example, a pitch efficiency of 75% or greater. Such a pitch efficiency can allow the lead nut 148 to be back-driven along the lead screw 130. The lead screw 130 can also be Teflon-coated to reduce friction and improve efficiency of its operation. The bearing 154 and the thrust bearings 156 are coupled to a distal end portion of the lead screw 130, and can at least partially support the lead screw 130 within the housing 140.

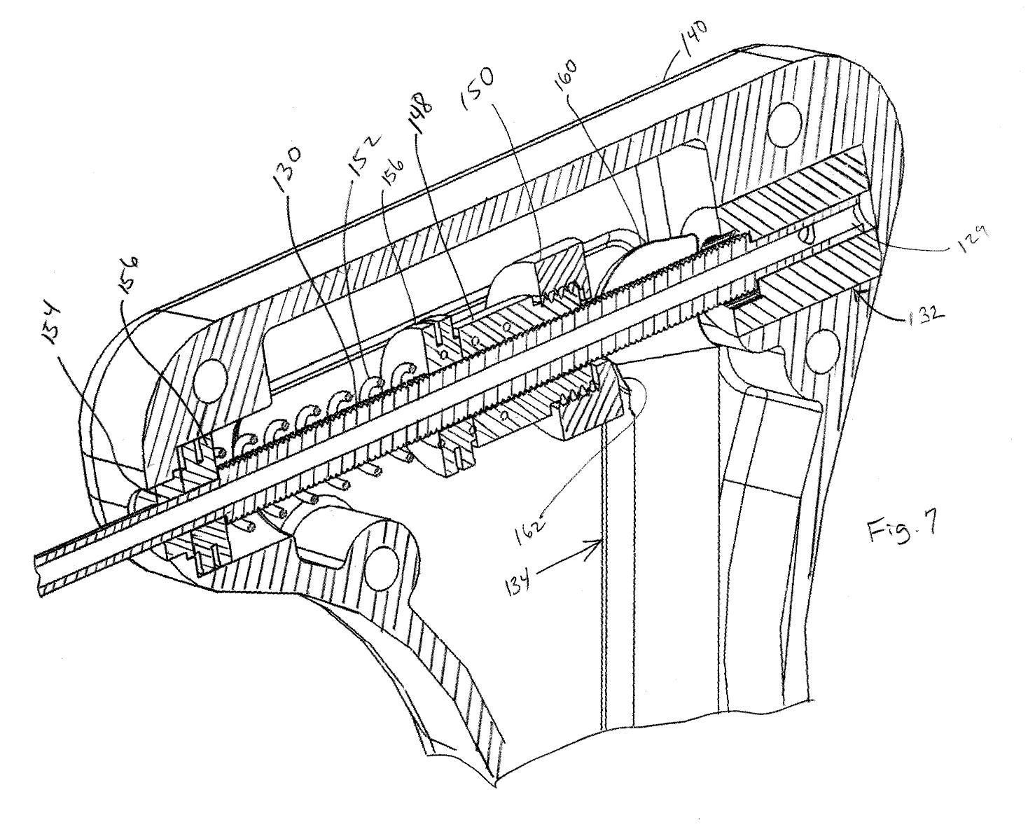

The rotation-limiting mechanism 132 can be coupled to a proximal end portion of the lead screw 130 and can at least partially support the lead screw 130 within the housing 140. As shown in FIG. 7, a proximal end portion 129 of the lead screw 130 can be disposed within an interior lumen of the rotation-limiting mechanism 132. In one embodiment, rotation-limiting mechanism can be a roller clutch that includes needle bearings on an inner surface of the roller clutch. The proximal end portion 129 of the lead screw 130 can be heat treated or hardened such that the needle bearings can engage an outer surface of the lead screw 130. The needle bearings are configured to engage the outer surface of the lead screw 130 such that the lead screw 130 can rotate in one direction (e.g., clockwise), but is held rotatably fixed in an opposite direction (e.g., counter-clockwise).

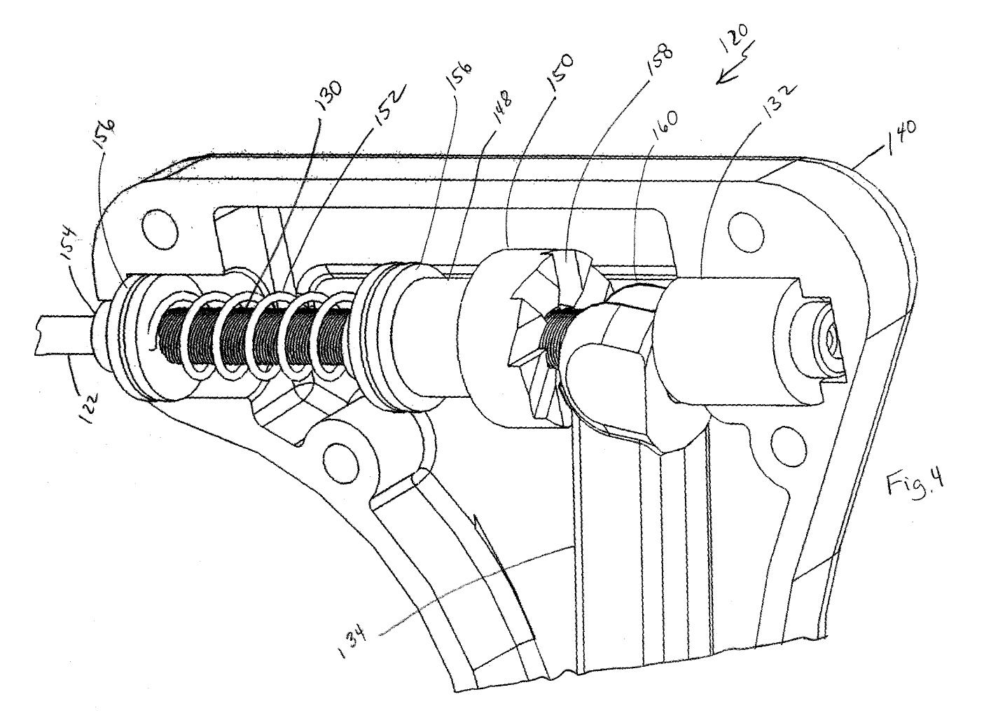

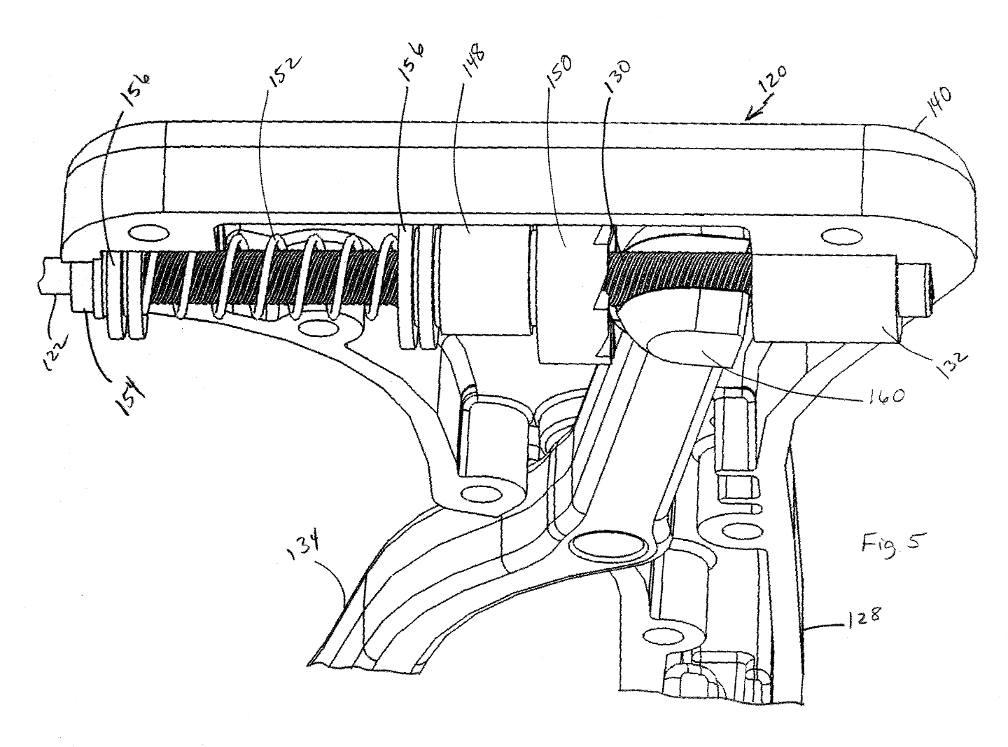

The lead nut 148 is disposed along a threaded portion of the lead screw 130 and has substantially the same pitch and thread form of the lead screw 130 such that the lead screw 130 can threadedly rotate relative to the lead nut 148. The face gear 150 is coupled to a proximal end of the lead nut 148. The face gear 150 has multiple teeth 158 that form an asymmetric tooth pattern as best shown in FIG. 4. The face gear 150 forms part of the one-way clutch system used to generate rotary motion, as described in more detail below. A top portion 160 of the lever 134 straddles the lead screw 130, as shown in FIG. 5 without rotatably engaging the lead screw 130. The top portion 160 of the lever 134 includes a protruding tooth 162 (see e.g., FIG. 3) that interfaces with the teeth 158 of the face gear 150. The protruding tooth 162 has a profile such that in one direction, the protruding tooth 162 mates with the teeth 158 of the face gear 150, thereby holding the face gear 150 and attached lead nut 148 rotationally fixed. In the opposite direction, the profile of the protruding tooth 162 and the profile of the teeth 158 of the face gear 150 allow the face gear 150 (and attached lead nut 148) to rotate during a user's release of the lever 134. For example, the protruding tooth 162 can engage a single tooth 158 of the face gear 150 when the lever 134 is actuated, but can disengage over multiple teeth 158 of the face gear 150 when the lever 134 is released, resetting the medical device 120. Thus, the face gear 150 and lever 134 form a type of one-way ratcheting mechanism.

The elongate member 122 is coupled to a distal end of the lead screw 130, and the return spring 152 is disposed about the distal end portion of the lead screw 130, as shown in FIGS. 3-5. The return spring 152 pushes against the lead nut 148 to reset the one-way mechanism. In other words, the return spring 152 biases the lead nut 148 in a proximal direction such that after a user squeezes the lever 134, the elongate member 122 completes a cycle of rotation (or a portion thereof) and the user releases the lever 134, the spring 152 will bias the lead nut 148 proximally. The medical device 120 will then be in a position such that it can be actuated again by squeezing the lever 134. Thus, the term “reset position” is used herein to mean the medical device 120 is in a position in which it is ready to actuate (e.g., the lever 134 is not squeezed).

For example, with the medical device 120 in a reset position (e.g., the return spring 152 has biased the lead nut 148 fully proximal within its range of motion, and the top portion 160 of the lever 134 is fully proximal within its range of motion), the user can actuate the medical device 120 by squeezing the lever 134 toward the handle 128. As the lever 134 is squeezed, the protruding tooth 162 on the top portion 160 of the lever 134 engages the gear teeth 158 on the face gear 150. The face gear 150 and attached lead nut 148 are held rotationally fixed by the engagement of the protruding tooth 162 to the teeth 158, but the actuation of the lever 134 translates the lead nut 148 in a distal direction. As a result of the lead nut 148 being rotationally fixed, yet being translated by the lever 134, the lead screw 130 is forced to rotate based on the pitch of the lead nut 148 and lead screw 130. Rotary motion occurs in a single direction (e.g., either clockwise or counter-clockwise) along the length of the lead screw 130 and along the elongate member 122 which is coupled to the distal end portion of the lead screw 130. As the user squeeze moves towards an end of its travel (i.e., range of motion) and while the lead screw 130 rotates, the return spring 152 will compress, and the top portion 160 of the lever 134 will be at a fully distal position, as shown in FIG. 7.

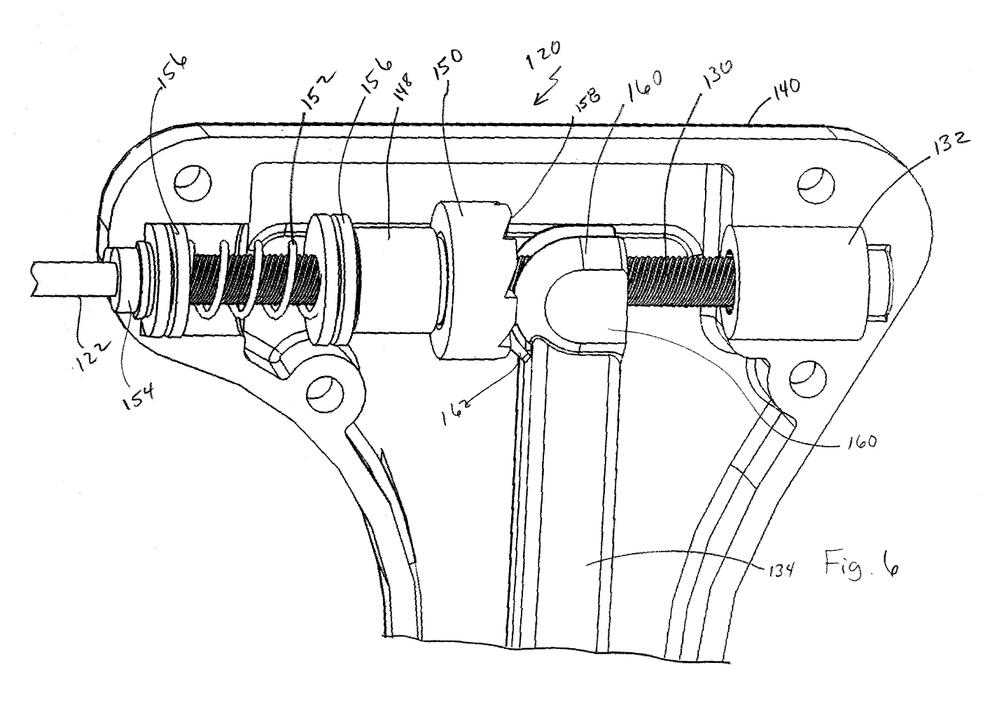

When the user releases the lever 134, the return spring 152 pushes back against the lead nut 148 as described above; however, the rotation-limiting mechanism 132 supporting the proximal end portion of the lead screw 130 does not allow the lead screw 130 to rotate in an opposite direction (e.g., opposite direction from its direction of rotation described above). Thus, the respective profiles of the protruding tooth 162 and the teeth 158 on the face gear 150 allow for relative rotation in a single direction. As the return spring 152 pushes against the lead nut 148, the lead nut 148 rotates and translates along the lead screw 130 back to its starting position (e.g., fully proximal). During this return sequence, the lead screw 130 is held rotationally fixed by the rotation-limiting mechanism 132. At this point, the medical device 120 is again back to a fully reset position (as shown in FIGS. 2-6) and ready for actuation of another cycle. The medical device 120 can be actuated several times consecutively to achieve a pulsed, rotary motion in a single direction.

As the lead screw 130 is rotated when a user squeezes the lever 134 as described above, the elongate member 122 coupled to the lead screw 130 will also rotate. The elongate member 122 can be configured with a variety of different tools to perform a variety of different medical procedures, such as, for example, tissue scraping, cutting, curetting and/or disrupting. As shown in FIG. 2, the elongate member 122 includes an expandable member 126 disposed at a distal end portion of the elongate member 122. As best shown in FIG. 8, the expandable member 126 includes multiple arms or tines 164, formed for example, by laser cutting longitudinal slits along a wall of the elongate member 122. The expandable member 126 and the elongate member 122 can be formed with, for example, a shape-memory material (e.g., nitinol or superelastic nitinol) such that the arms 164 of the expandable member 126 can be biased into an expanded configuration. Each of the arms 164 when in the expanded configuration are curved or flared in a lengthwise or longitudinal direction, but in other embodiments, the arms 164 can be substantially straight in a longitudinal direction, and/or have other shapes and/or configurations.

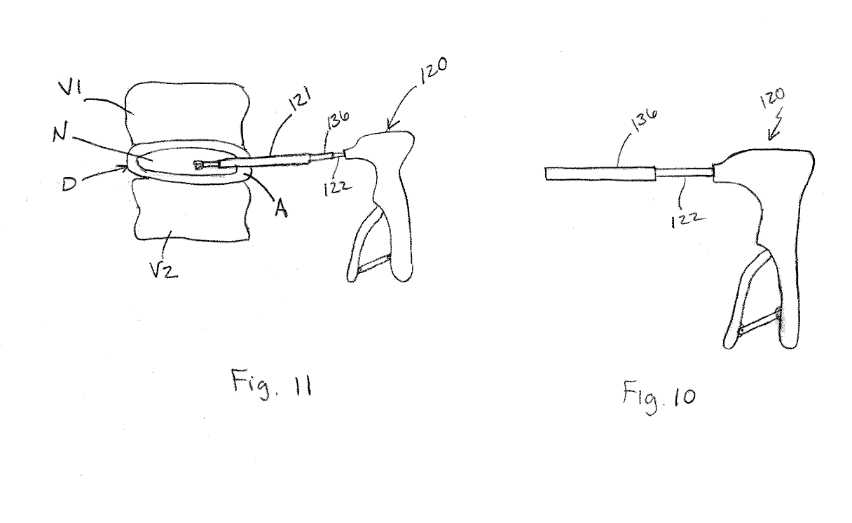

The expandable member 126 can be moved from the expanded configuration to a collapsed configuration (not shown). For example, the expandable member 126 can be restrained within an access cannula or an optional sheath 136 (see FIG. 10) for insertion into a biological body or tissue. The sheath 136 can be slidably placed over the elongate member 122 such that a user of the medical device 120 can manually slide the sheath 136 relative to the elongate member 122. For example, the sheath 136 can be moved in a distal direction until the expandable member 126 is disposed within a lumen of the sheath 136 as shown in FIG. 10. This relative movement of the sheath 136 will move the expandable member 126 from its biased expanded configuration to its collapsed configuration. The sheath 136 can be moved proximally such that the expandable member 126 exits a distal end of the sheath 136 and the expandable member 126 can assume its biased expanded configuration. The expandable member 126 in its expanded configuration defines an interior region 163 that is in communication with a lumen (not shown) of the elongate member 122. The expandable member 126 in its expanded configuration has a greater size than an outer diameter of the elongate body 122.

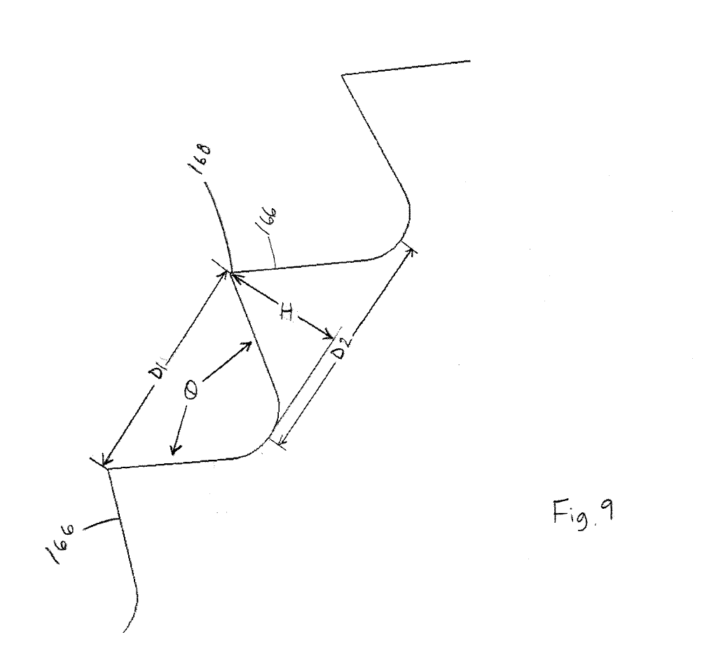

The arms 164 can each include a cutting portion along an edge of the arms 164. For example, the arms 164 can have a sharpened edge or, as shown in FIG. 8, the arms 164 can include serrations 166 along an edge of the arms 164. In alternative embodiments, the arms can include serrations only on a portion of the edge of the arms, for example, along a leading edge of the arms in a direction of rotation. The serrations 166 (also referred to herein as “teeth”) can be formed by laser cutting, for example, when the arms 164 are laser cut in the side wall of the elongate member 122. As shown in FIG. 9, each individual serration 166 can have, for example, a height H that is 1/10^{th} a width (not shown in FIG. 9) of the particular arm 164 on which the serration is formed. The distance or spacing D1 between individual serrations 166 measured from peak-to-peak can substantially equal, for example, a distance D2 measured valley-to-valley between the serrations. An angle θ between the edges of consecutive serrations 166 can be, for example, 60 degrees. An end portion 168 of the serrations 166 can be, for example, substantially flat or linear, can form a sharp tip (as shown in FIG. 9), or can be rounded or curved.

The medical device 120 can be used for a variety of different types of medical procedures. An example use of the medical device 120 is described below with reference to expandable member 126 and elongate body 122, but it should be understood that the medical device 120 can include expandable member 226 (and corresponding elongate body 222) or other variations of a tissue interaction member.

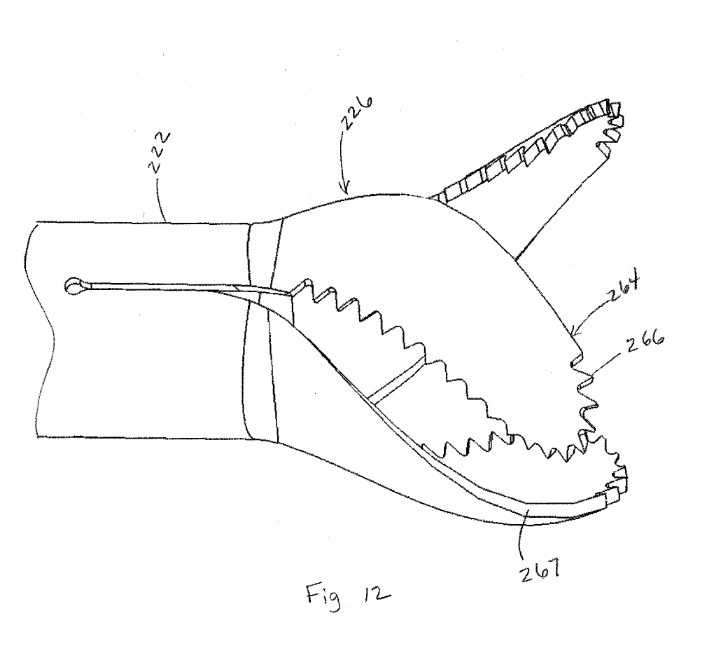

In one example, the medical device 120 can be used to treat a herniated intervertebral disc. For example, the medical device 120 can be used to disrupt and remove nucleus material from an interior of an intervertebral disc. An access path into the intervertebral disc can be made, for example, with a stylet or other access tool through, for example, Kambin's triangle. An optional access cannula 121 (shown in FIG. 11) can be inserted into an intervertebral disc D (shown in cross-section disposed between a vertebra V1 and a vertebra V2) via the access path. The access cannula 121 is inserted through the annulus of the intervertebral disc D and its distal end is disposed within the nucleus N of the intervertebral disc (e.g., just inside the annular wall). The medical device 120 can then be inserted through a lumen of the access cannula 121. For example, as described above the sheath 136 can be placed over the expandable member 126 to collapse the expandable member 126 (as shown in FIG. 10). The medical device 120 can then be inserted through the lumen of the cannula 121 and into the nucleus N of the intervertebral disc D. When a distal end of the medical device 120 is in a desired position within the intervertebral disc D, the sheath 136 can be moved proximally relative to the elongate member 122 such that the expandable member 126 is unrestrained and can move to its expanded configuration as shown in FIG. 11.

With the expandable member 126 in its expanded configuration, the medical device 120 can be actuated as described above to rotate the elongate member 122 and expandable member 126 within the nucleus N of the intervertebral disc D. As the expandable member 126 rotates, the serrations 166 on the arms 164 will cut, tear or otherwise disrupt tissue within the nucleus N of the intervertebral disc D. The medical device 120 can be actuated once, or repeatedly to generate pulses of rotation. The medical device 120 can also be translated proximally and distally while the expandable member 126 is rotated. Such translation can form a channel of disrupted nucleus material within the intervertebral disc D.

When the user (e.g., medical practitioner) is satisfied with the amount of tissue that has been disrupted, the medical device 120 is removed from the disc. For example, the medical device 120 can be pulled proximally, such that the expandable member 126 is pulled into the lumen of the access cannula 121 and is moved to the collapsed configuration. Alternatively, the sheath 136 can be moved distally over the expandable member 126 and relative to the elongate member 122 to collapse the expandable member 126. In either case, with the expandable member 126 in the expanded configuration, the medical device 120 is removed from the disc D through the lumen of the access cannula 121.

To remove the disrupted nucleus material from within the intervertebral disc D, suction can be applied to draw the disrupted nucleus material through the lumen of the access cannula 121. For example, a suction source (not shown) can be coupled to a proximal end of the cannula 121 and used to provide suction within the lumen of the access cannula 121. Alternatively, a separate suction tool (not shown) can be inserted through the lumen of the access cannula 121 and used to suction nucleus material out of the intervertebral disc D and to a location outside of the patient. A saline solution can optionally be flushed through the lumen of the access cannula 121 prior to suctioning the disrupted nucleus material to mobilize the disrupted material. The optional flushing and suctioning can be repeated as necessary to remove the disrupted nucleus material.

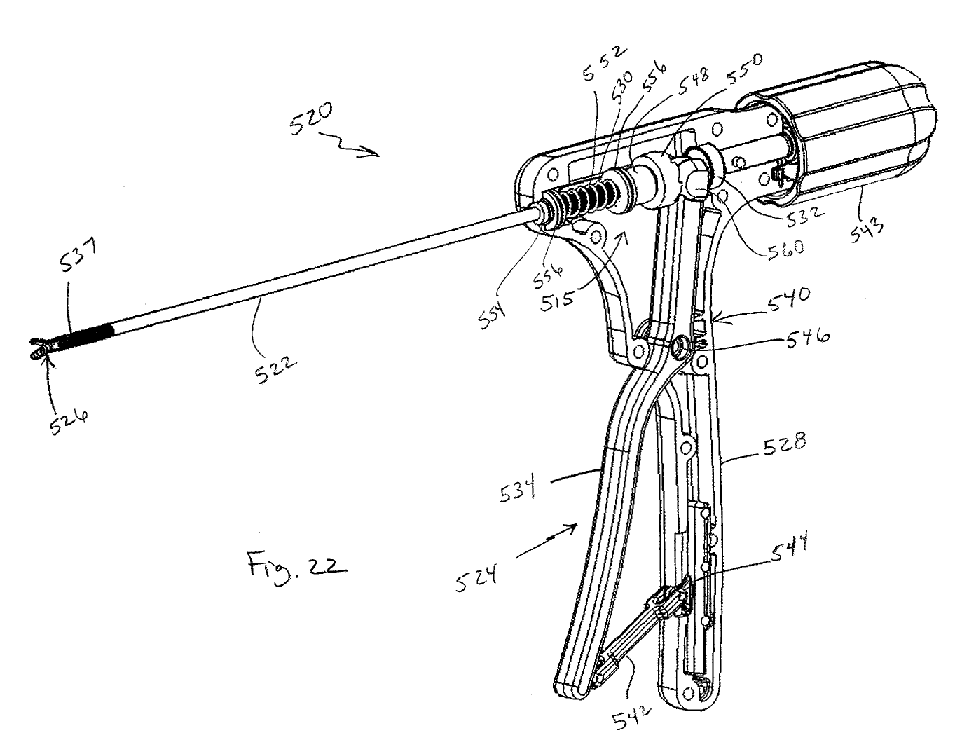

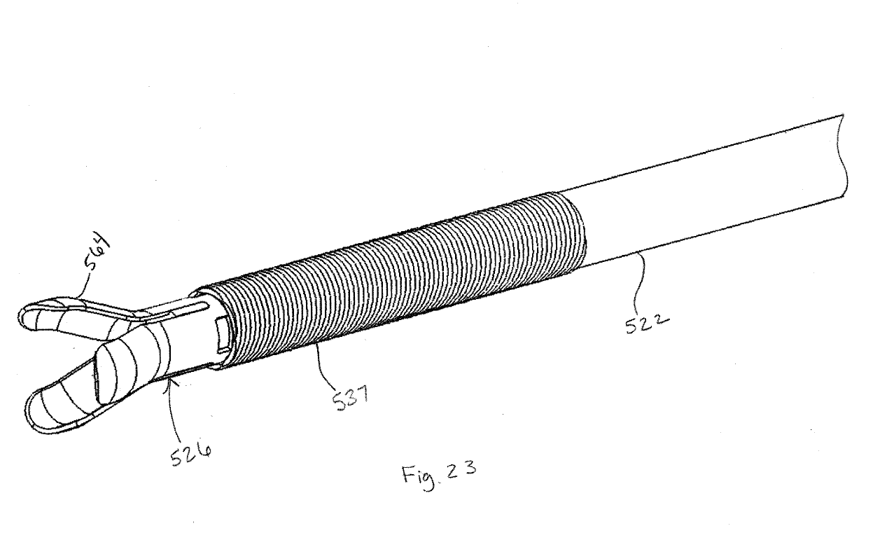

In an alternative embodiment, the irrigation and suction functions can be incorporated within the medical device 120. For example, the lumen of the elongate member 122 can be in communication with a lumen defined by the lead screw 130 to collectively define a passageway through the medical device to an opening on a proximal end of the medical device 120. A source of fluid (e.g., saline solution) can be coupled to the medical device 120 to provide a saline flush through the medical device 120 and into the intervertebral disc before, during or after the disruption procedure has been performed. A source of suction can also be coupled to the medical device 120 in the same manner. Such an embodiment is illustrated with reference to FIGS. 20 and 21, which are discussed below.

In some embodiments, the expandable member 126 can be used to remove the disrupted nucleus material. The expandable member 126 can be moved to the collapsed configuration within the nucleus by moving the access cannula distally over the expandable member 126, such that disrupted nucleus material is captured within the interior region 163 of the expandable member 126. The medical device 120 can be withdrawn with the captured disrupted material.

The expandable member 126 (and also expandable member 226 discussed below in connection with FIG. 12) can alternatively be coupled to other types of medical devices and used to cut, tear or otherwise disrupt tissue as described above. For example, the expandable member 126 can be coupled to or incorporated with an elongate member that is coupled to an automated rotary device, rather than the manual actuation described above. The expandable member 126 can also be used independently in that it can be used without providing a mechanism to rotate the expandable member 126. The expandable member 126 can be inserted into a biological body and actuated between a collapsed configuration and expanded configuration using a cannula or sheath as described above.

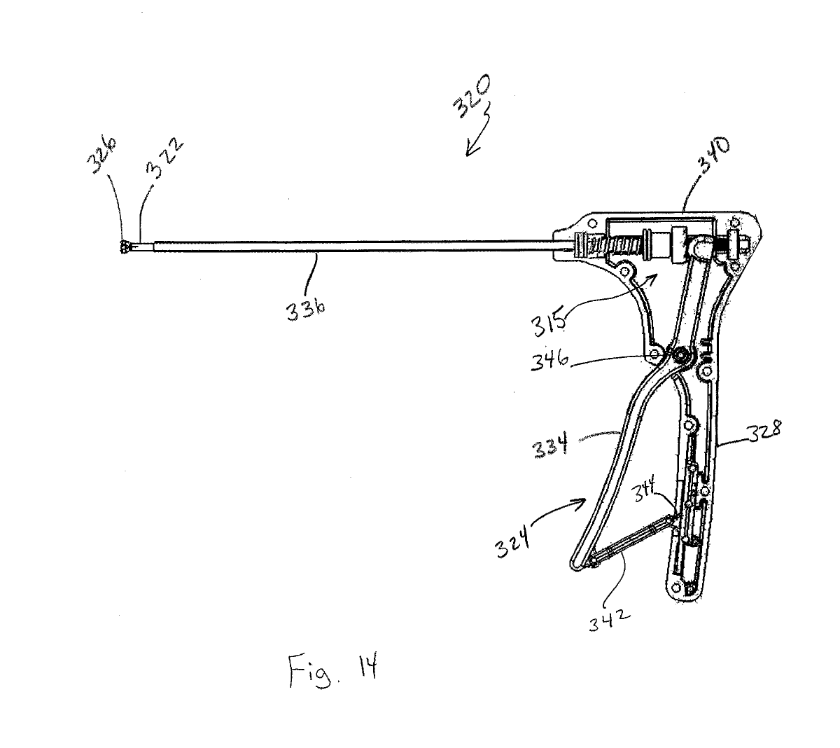

In an alternative embodiment, shown in FIGS. 12 and 13, an expandable member 226 can have a spiral configuration. As with the expandable member 126, the expandable member 226 includes arms 264 formed, for example, by slits cut (e.g., laser cut) along a side-wall of an elongate member 222. The expandable member 226 can be formed with a nitinol or superelastic nitinol shape-memory material that is heat-set into the spiral configuration. Thus, the expandable member 226 has a biased expanded configuration as shown in FIGS. 12 and 13. The arms 264 can also have a curved or flared configuration as shown in FIG. 12. In this embodiment, the arms 264 include serrations 266 along only a leading edge of the arms 264. In this example embodiment, the elongate member 222 and expandable member 226 are configured to rotate in a clock-wise direction as indicated by the leading edge on which the serrations 266 are disposed. The serrations 266 can be sized and configured in the same manner as described above with reference to serrations 166 (see FIG. 9). It is to be understood that in some embodiments the arms can also include serrations along a trailing edge of the arms in addition to the leading edges. For example, in some embodiments, the arms can include serrations along an entire edge of the arms.

The expandable member 226 can be moved from the expanded configuration to a collapsed configuration. As described above for expandable member 126, the expandable member 226 can be restrained within an access cannula or sheath (not shown in FIGS. 12 and 13) for insertion into a biological body or tissue. When the expandable member 226 exits a distal end of the cannula, the expandable member 226 can assume its biased expanded configuration. As shown in FIG. 12, the expandable member 226 in its expanded configuration has a greater size than an outer diameter of the elongate body 222. FIG. 13 is a distal end view of the expandable member 226 (shown without serrations 266 for illustration purposes) in its expanded configuration. As shown in FIG. 13, the arms 264 define an interior region 263 that is in communication with a lumen 225 of the elongate member 222. FIG. 13 also illustrates a flared configuration of the arms 264 that is counterclockwise corresponding to a clockwise rotation of the spiral configuration. Alternatively, arms can be formed to flare clockwise if an opposite drive direction (e.g., a counterclockwise direction) is desired.

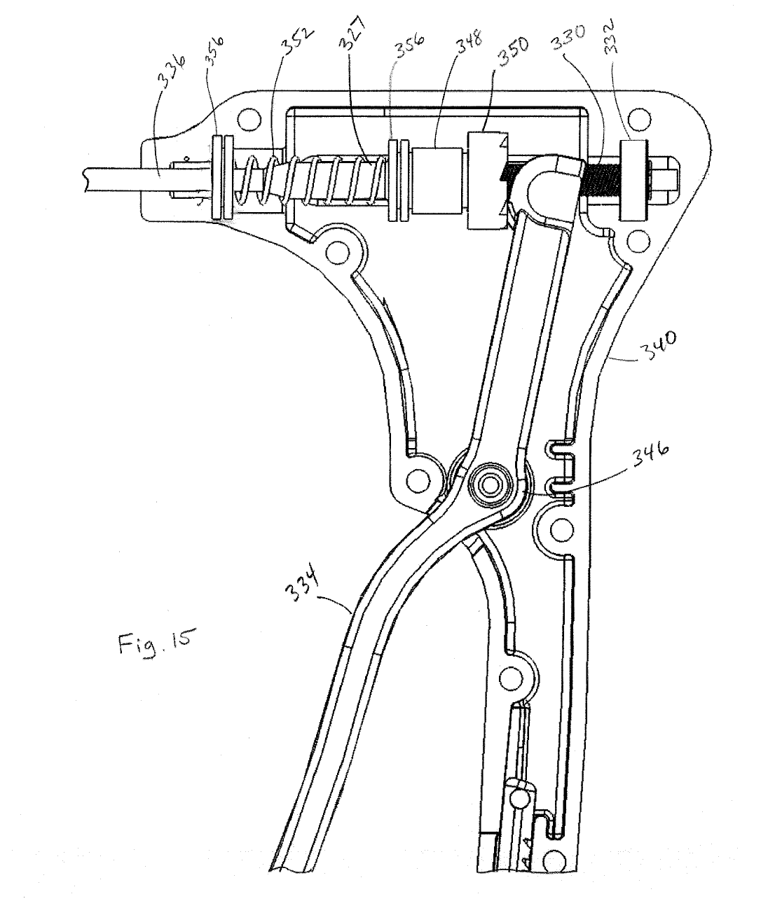

FIGS. 14-19 illustrate another embodiment of a medical device that includes a translating sheath that can be actuated by actuation of the medical device. A medical device 320 includes an elongate member 322 coupled to a conversion mechanism 315, which is coupled to an actuation mechanism 324 and a housing 340. An expandable tissue interaction member 326 (referred to herein as expandable member 326) is disposed at a distal end of the elongate member 322. The expandable member 326 includes arms 364 and has a biased expanded configuration. The expandable member 326 (and arms 364) can be moved to a collapsed configuration as described above for expandable members 126 and 226. The expandable member 326 can include serrations (not shown) and can be used to tear, cut, or otherwise disrupt tissue as previously described.

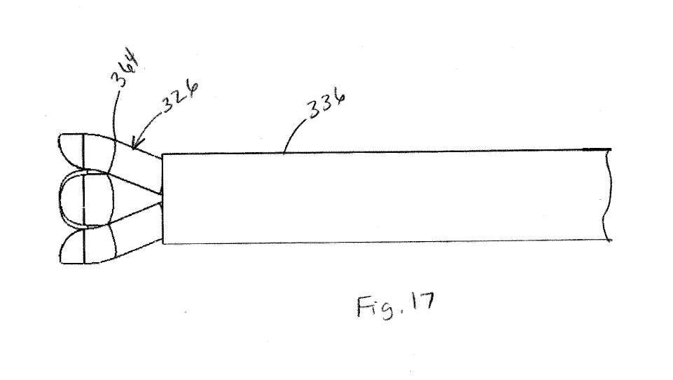

A translating sheath 336 is disposed at least partially over the elongate member 322 and is coupled to the actuation mechanism 324. As with the previous embodiment, the housing 340 includes a handle 328 and the actuation mechanism 324 includes a lever 334. The lever 334 is coupled to the housing 340 via a pivot arm 342 (see FIG. 16) and also at a pivot joint 346. The pivot arm 342 is coupled to a slide member 344.

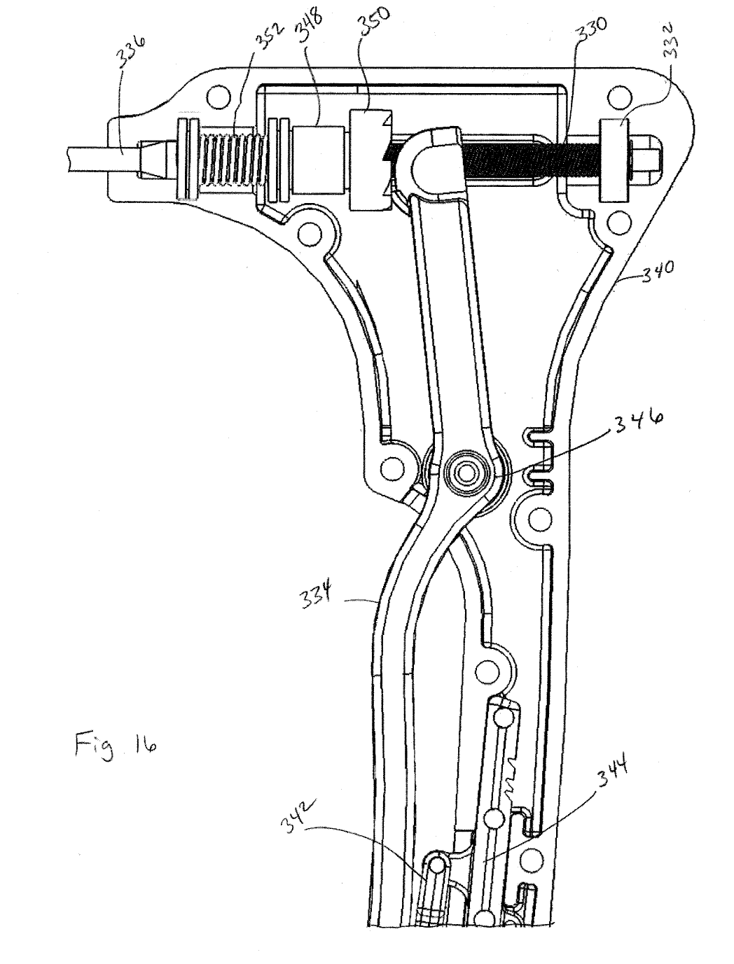

As shown in FIGS. 15 and 16, the conversion mechanism 315 includes a lead screw 330, a roller clutch 332, a lead nut 348, a face gear 350, a return spring 352, and a pair of thrust bearings 356 similar to the conversion mechanism 115 described above. The conversion mechanism 315 and actuation mechanism 324 function in a similar manner as described above for conversion mechanism 115 and actuation mechanism 124 to mechanically transform translational motion into rotary motion of the elongate member 322, and therefore, will not be described in detail with reference to this embodiment. In this embodiment, the actuation mechanism 324 also functions to translate the sheath 336 proximally and distally while simultaneously actuating the conversion mechanism 115 to rotate the elongate member 322.

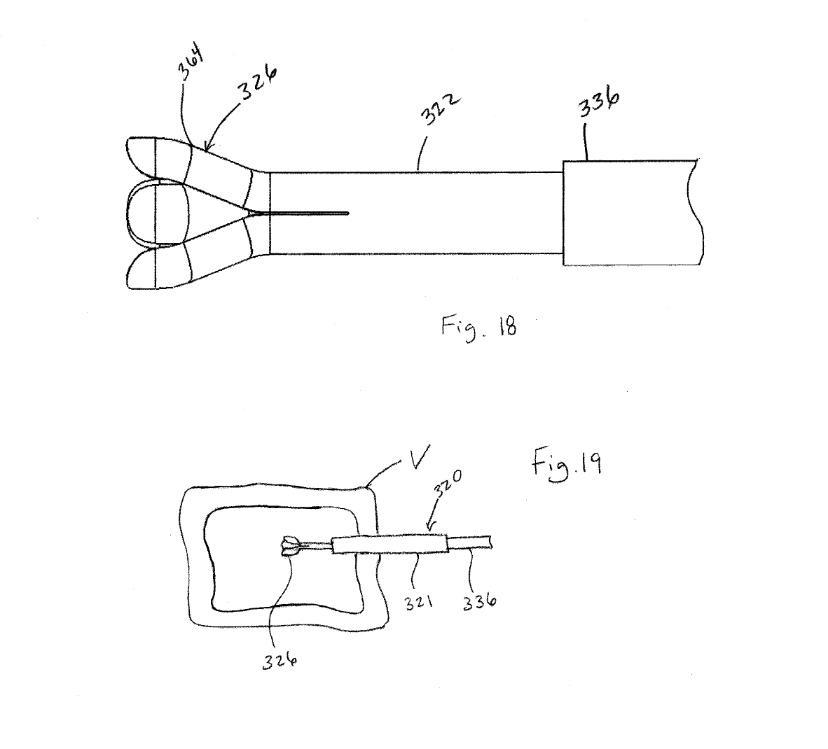

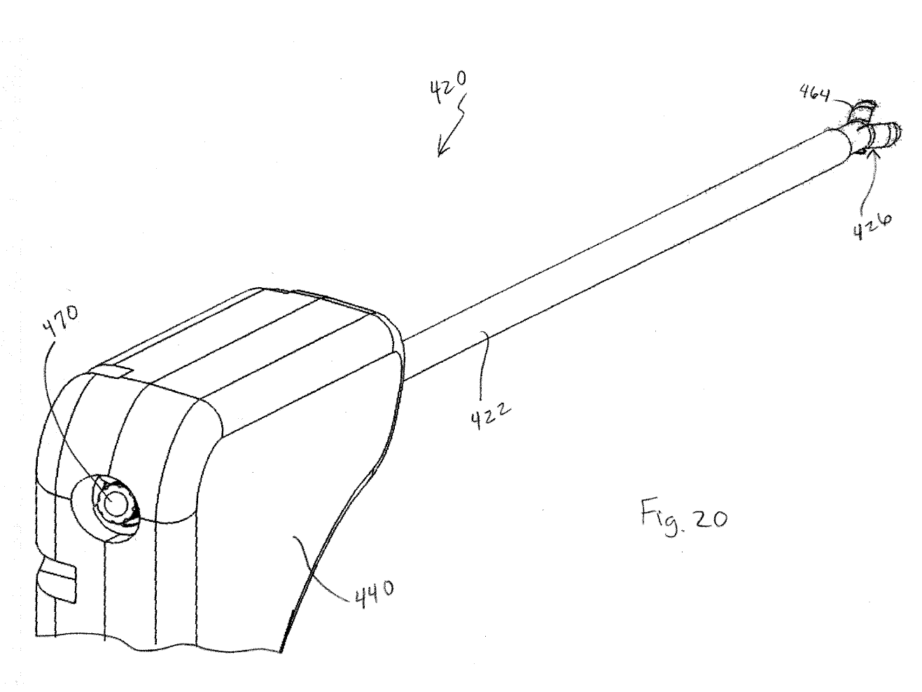

When the actuation mechanism 324 is actuated (e.g., lever 334 is squeezed), the translational motion of the lever 334 and the lead nut 348 are transformed into rotary motion of the elongate member 322 as described above. A distal end portion 327 of the sheath 336 extends through an interior region defined by the return spring 352 and is coupled to the lead nut 348 such that when the actuation mechanism 324 is actuated, the sheath 336 is moved distally. Near the completion of the rotational cycle of the elongate member 322, the sheath 336 will reach a distal end portion of the elongate member 322 and be disposed at least partially over the expandable member 326, thereby collapsing the expandable member 326. FIG. 15 illustrates the actuation mechanism 324 and conversion mechanism 315 in a reset position with the lead nut 348 and sheath 336 in a proximal position (e.g., ready to be actuated), and the return spring 352 in an uncompressed position. FIG. 16 illustrates the actuation mechanism 324 and conversion mechanism 315 after being actuated, with the lead nut 348 and sheath 336 translated distally, and the return spring 352 in a compressed position. FIG. 17 illustrates the expandable member 326 partially collapsed within a distal end portion of the sheath 336, for example, when the sheath 336 begins to move distally relative to the elongate member 322 and is starts to collapse the expandable member 326. FIG. 18 (and also FIG. 14) illustrates the expandable member 326 in its expanded configuration disposed outside of the sheath 336, for example, when the sheath 336 is moved proximally relative to the elongate member 322 and is no longer covering the expandable member 326.

As with the previous embodiments, the medical device 320 can be used, for example, to cut, tear, disrupt or debulk tissue. The medical device 320 can be used to disrupt tissue within an intervertebral disc as described above. The medical device 320 can also be used in conjunction with an access cannula as described above (e.g., cannula 121 shown in FIG. 11). In another example use, the medical device 320 can be used, for example to perform a bone biopsy procedure. The medical device 320 can be actuated using a single hand of the user, and can debulk and remove tissue fragments without repeated tool insertions and withdrawals. Thus, the debulked and/or disrupted tissue fragments can be captured and removed with the medical device 320 with a single actuation of the medical device 320 while the expandable member 326 is disposed within a biological body.

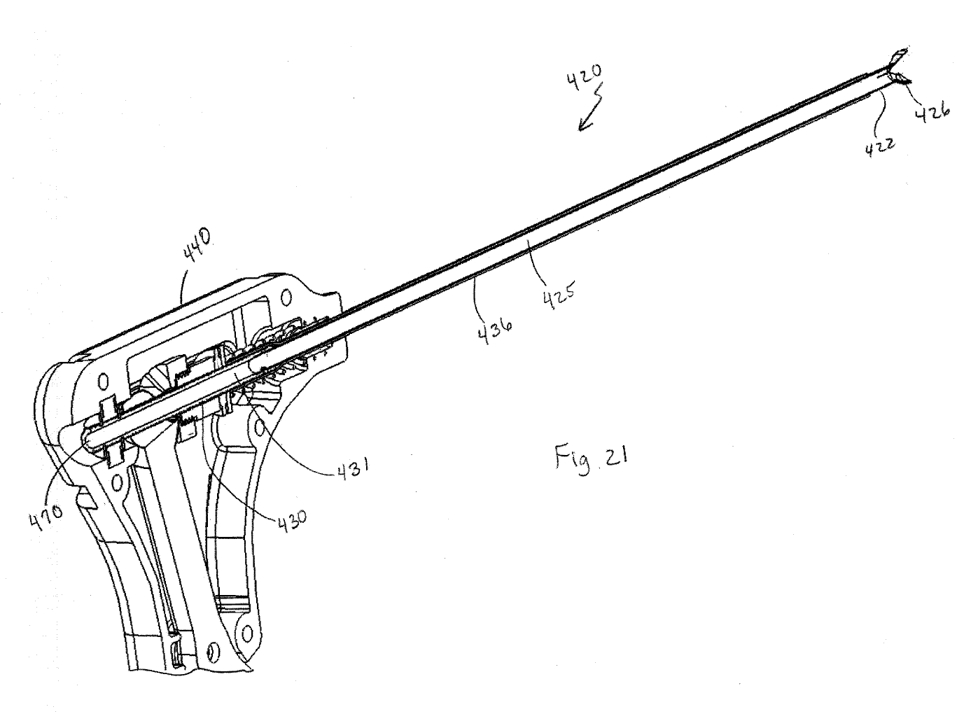

For example, as shown in FIG. 19, an access cannula 321 can be inserted into a vertebra V. The medical device 320 can alternatively be inserted through an access opening made, for example, with a stylet or other tool. The medical device 320 can be actuated prior to insertion into the patient's body (e.g., prior to insertion through the cannula 321) such that the expandable member 326 is moved to a collapsed configuration. For example, as described above, the actuation mechanism 324 can be actuated by squeezing the lever 334, which will cause the sheath 336 to be moved distally over the expandable member 326. As the lever 334 is held in a fully squeezed position, a distal end of the medical device 320 is inserted through the lumen (not shown) of the access cannula 321 and into an interior of the vertebra V. The user can then release the lever 334 such that the sheath 336 translates back to a reset position (e.g., fully proximal), and the expandable member 326 can move to its expanded configuration.

With the expandable member 326 in the expanded configuration, the expandable member 326 can be advanced to a desired tissue site within the vertebra V. The lever 334 can then be actuated a second time, which will cause the elongate member 322 and expandable member 326 to rotate to disrupt tissue within the vertebra V. As the actuation nears an end of the cycle, the sheath 336 translates over the expandable member 326, and the expandable member 326 collapses over a portion or fragment of the disrupted tissue. The tissue fragment is captured within an interior region (not shown) defined by the expandable member 326 and sequestered from the remaining portion of tissue within the vertebra V. The medical device 320 can then be removed from the vertebra V and the access cannula 321, with the tissue fragment captured therein.

With the medical device 320 outside of the patient's body, the user can release the lever 334 such that the sheath 336 is translated proximally, and the expandable member 326 is moved to the expanded configuration. The tissue fragment can then be removed from the medical device 320. In some embodiments, suction force can be used to draw the tissue fragments through a lumen of the elongate member 322. An example of such an embodiment is described below with reference to FIGS. 20 and 21. The above procedure can be repeated as necessary for further debulking or disrupting and tissue removal.

The medical device 320 can also be used in a similar manner as a bone biopsy device. The medical device 320 can be actuated such that rotation of the expandable member 326 aids in coring a bone sample; the sheath 336 then translates over the expandable member 326 with the bone sample captured therein. The medical device 320 can be removed from the biological body with the core sample disposed within the interior region of the expandable member 326. Such a biopsy procedure can be performed in hard tissue areas, such as within a bone structure (e.g., a vertebra), or soft tissue areas, such as within an intervertebral disc.

FIG. 20 illustrates a portion of a medical device according to another embodiment. This embodiment is constructed similar to the medical device 320 described above, and that can perform the same functions as described above. Thus, details of various common or similar components are not described with reference to this embodiment. In this embodiment, a medical device 420 includes an elongate member 422 that is partially disposed through the medical device 420 with a proximal end of the elongate member 422 being proximate to a proximal end of a housing 440. For example, a lead screw 430 (shown in FIG. 21) defines a lumen 431 through which the elongate member 422 can partially extend. A lumen 425 of the elongate member 422 and the lumen 431 of the lead screw 430 are collectively in fluid communication with a port 470 defined by the housing 440. A suction line (not shown in FIG. 20) can be coupled to the port 470 to allow for tissue fragments to be suctioned through the medical device 420 and into a containment reservoir (not shown). A suction force can be applied while the medical device 420 is actuated within a biological body, or after the medical device 420 has been removed from a patient with a tissue fragment captured within an expandable member 426 of the medical device 420. The port 470 can also be used for introducing a fluid such as a saline solution through the medical device and into the biological body. Such irrigation can be performed before, during or after the medical device 420 dubulks or disrupts tissue.

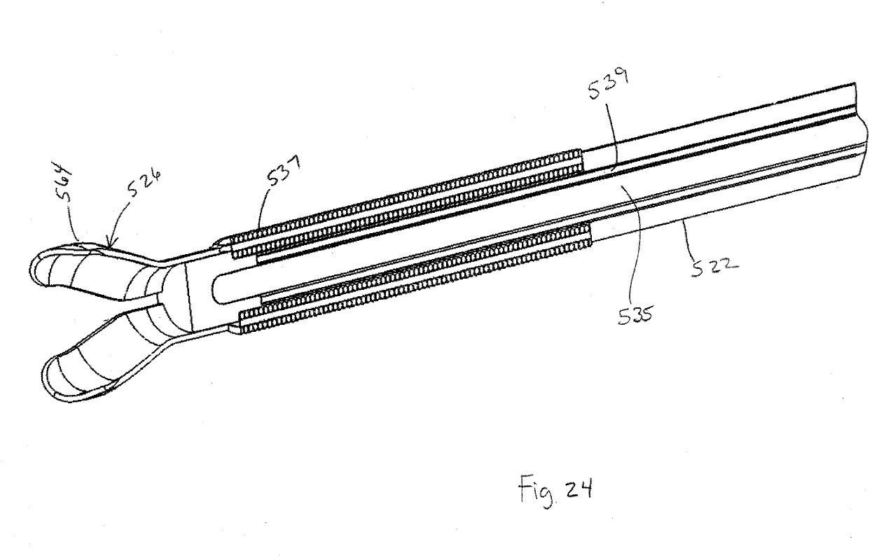

FIGS. 22-28 illustrate an embodiment of a medical device having a steering mechanism configured to steer a distal end portion of the medical device within a biological body. A medical device 520 includes an elongate member 522 coupled to conversion mechanism 515, which is coupled to an actuation mechanism 524 and a housing 540. An expandable tissue interaction member 526 (referred to herein as expandable member 526) is disposed at a distal end of the elongate member 522. As with the previous embodiments, the housing 540 includes a handle 528 and the actuation mechanism 524 includes a lever 534. The lever 534 is coupled to the housing 540 via a pivot arm 542 and also at a pivot joint 546. The pivot arm 542 is coupled to a slide member 544. Other components of the actuation mechanism 524 are disposed within the housing 540 as described below.

As shown in FIG. 22, the conversion mechanism 515 includes a lead screw 530, a roller clutch 532, a lead nut 548, a face gear 550, a return spring 552, a bronze bearing 554, and a pair of thrust bearings 556 similar to the conversion mechanism 115 and 315 described above. The lever 534 includes a top portion 560 coupled to the lead screw 530 also as described above. The conversion mechanism 515 functions in a similar manner as described above for the conversion mechanisms 115 and 315 to mechanically transform translational motion into rotary motion of the lead screw 530 and elongate member 522. Therefore, such functions are not described in detail with reference to this embodiment.

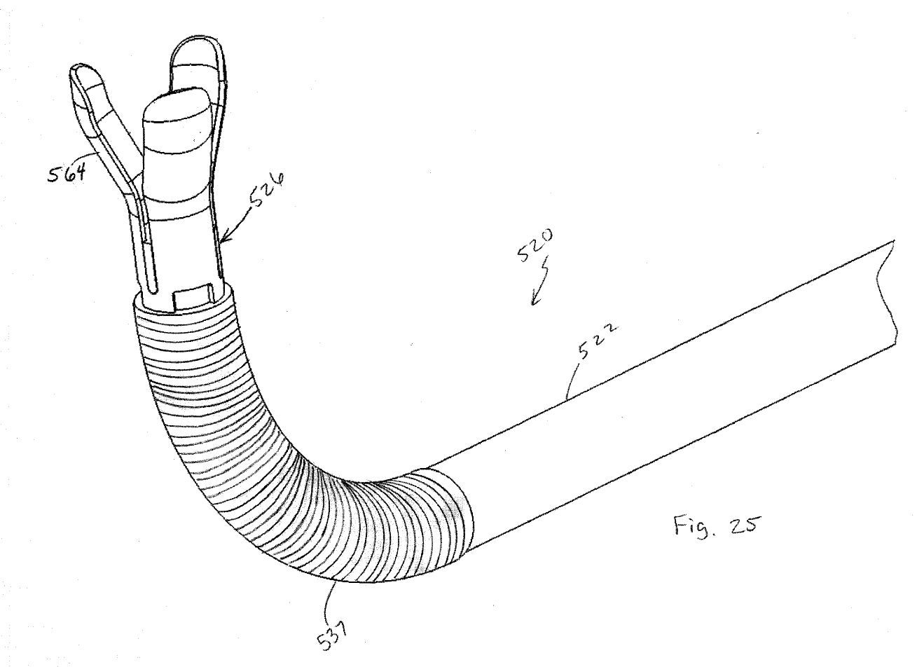

In this embodiment, a flexible member 537 is coupled to a distal end of the elongate member 522 and the expandable member 526 is disposed at a distal end of the flexible member 537, as best shown in FIGS. 23-26. The expandable member 526 includes arms 564, and has a biased expanded configuration. The expandable member 526 (and arms 564) can be moved to a collapsed configuration as described above (e.g., for expandable members 126, 226, 326, 426). The expandable member 526 can also include serrations (not shown), and can be used to tear, cut, or otherwise disrupt tissue as previously described. In this embodiment, the expandable member 526 is a separate component from the elongate member 522, but can be formed in a similar manner. For example, the arms 564 of the expandable member 526 can be formed by laser cutting longitudinal slits along a tubular component formed, for example, with a shape memory material. The arms 564 can then be heat-set into a biased expanded configuration.

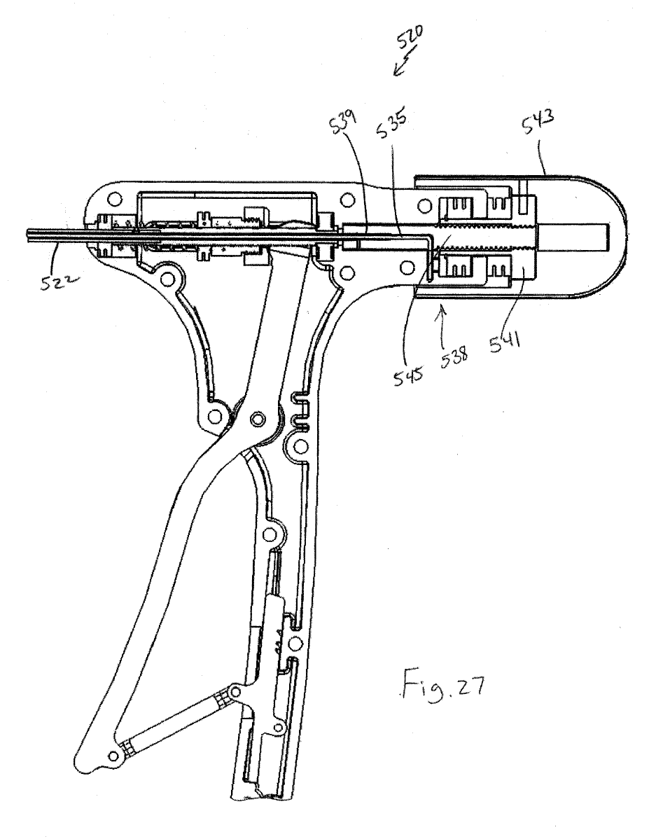

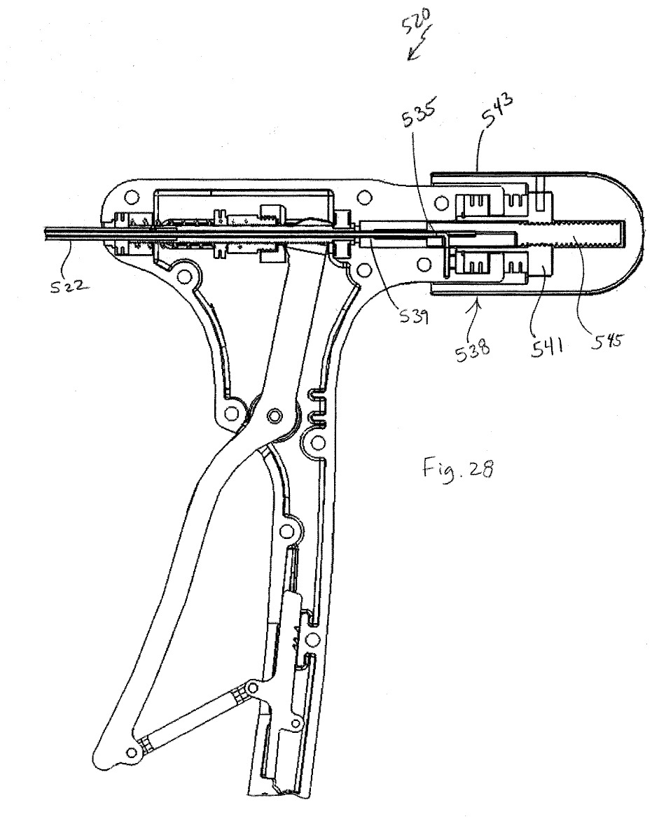

The flexible member 537 can be formed, for example, with a flexible cable material or spring material, such as a torque cable. In other embodiments, the flexible member 537 can be formed with a flexible material that has a substantially smooth surface. The flexible member 537 can alternatively be formed monolithically with the elongate member 522. The flexible member 537 is formed such that it can be moved between a substantially straight or linear configuration as shown in FIGS. 22-24 and a curved configuration as shown in FIGS. 25 and 26. The curvature of the flexible member 537 shown in FIGS. 25 and 26 is merely an example curvature, as the flexible member 537 can be reconfigured into multiple different curvatures as desired. The flexible member 537 is used in conjunction with a steering mechanism 538, which is used to move the flexible member 537 between the substantially linear configuration and the curved configuration to steer or maneuver a distal end portion of the medical device 520 within a biological body.

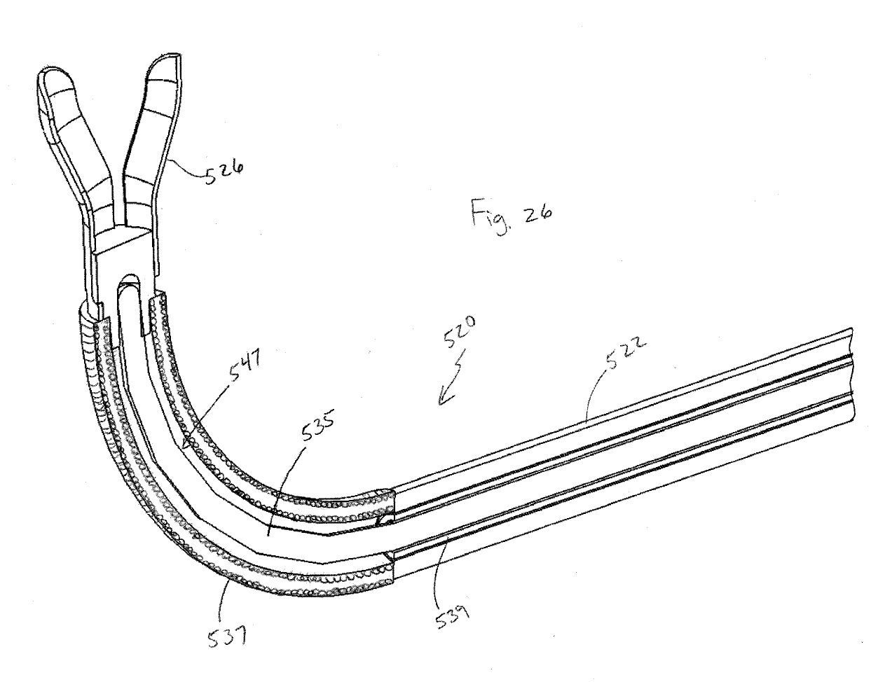

The steering mechanism 538 includes an elongate steering rod 535 disposed within a lumen of a restraining element. In this embodiment, the restraining element is a steering sheath or tube 539 as shown in the cross-sectional views of FIGS. 24 and 26. The steering tube 539 extends through a lumen of the elongate member 522 and a lumen of the flexible member 537. The steering rod 535 is formed of a shape-memory material, such as nitinol or superelastic nitinol (or any other type of material that can maintain a biased configuration), and a distal end portion 547 of the steering rod 535 is heat set into a biased curved configuration as shown in FIG. 26. When the distal end portion 547 of the steering rod 535 is restrained within the steering tube 539 it is moved to a different curvature than when the steering rod 535 is unconstrained. The steering rod 535 can have, for example, a substantially linear or straight configuration when constrained within the steering tube 539, as shown in FIG. 24. The amount of curvature of the steering rod 535 can depend on the amount or portion of the steering rod 535 that is constrained within the steering tube 539.

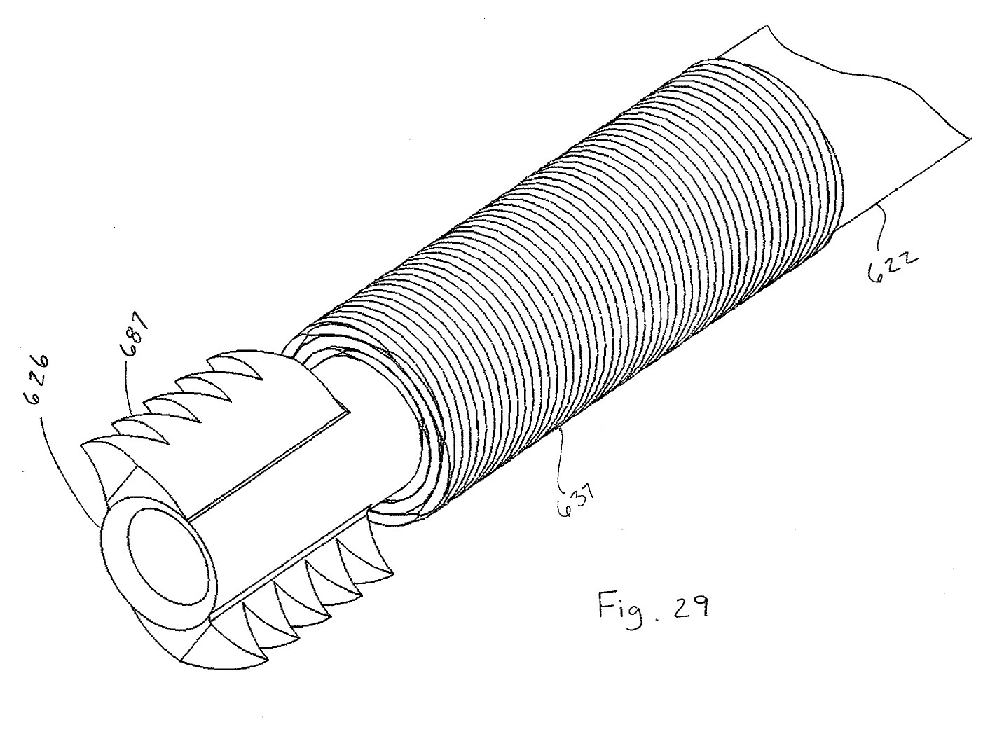

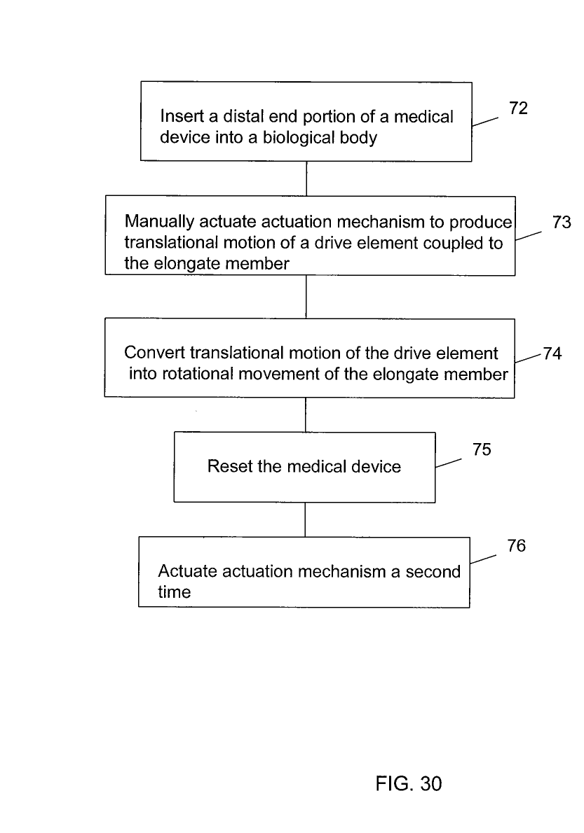

As shown in FIGS. 27 and 28, a distal end portion of the steering tube 539 is coupled to a threaded drive member 545, which is matingly (e.g., threadedly) coupled to a stationary drive nut 541. A steering knob 543 is matingly (e.g., threadedly) coupled to the stationary drive nut 541 and used to actuate the steering mechanism 538. To operate the steering mechanism 538, a user turns the steering knob 543 (e.g., clockwise or counter-clockwise about an axis substantially parallel to a longitudinal axis of the proximal end portion of the elongate member 522) and the steering knob 543 rotates the stationary drive nut 541, but the stationary drive nut 541 does not translate proximally or distally. Because the drive nut 541 is held stationary in a proximal-distal position, it causes the threaded drive member 545 to move proximally or distally (depending on which direction the steering knob 543 was rotated) relative to the drive nut 541. When the threaded drive member 545 moves, it in turn moves the steering tube 539 in the same direction (e.g., proximally or distally). For example, when the threaded drive member 545 is moved proximally to a position as shown in FIG. 28, the steering tube 539 will move proximally such that a distal end portion of the steering tube 539 is no longer covering the distal end portion 547 of the steering rod 535 as shown in FIG. 26. When the threaded drive member 545 is moved distally to a position as shown in FIG. 27, the steering tube 539 will be moved distally and be disposed over at least a portion of the distal end portion 547 of the steering rod 535.

Thus, when the steering knob 543 is moved clockwise, the steering tube 539 is moved proximally (e.g., toward the steering knob 543), and the distal end portion 547 of the steering rod 535 will be uncovered (no longer restrained within the lumen of the steering tube 539). With the distal end portion 547 of the steering rod 535 no longer constrained within the steering tube 539, it can move to its biased curved configuration as shown in FIG. 26 (or other curvature as desired). As the steering rod 535 is moved to its curved configuration, it will cause the flexible member 537 to also be moved to its curved configuration, as shown in FIGS. 25 and 26. When the steering knob 543 is moved counterclockwise, the steering tube 539 will be moved distally over at least a portion of the distal end portion 547 of the steering rod 535 as shown in FIG. 24, moving the steering rod 535 to a different curvature (e.g., substantially straight or linear configuration) and the flexible member 537 to its straight or linear configurations, as shown in FIGS. 23 and 24. The amount of curvature of the steering rod 535 and flexible member 537 will depend on the amount of rotation of the steering knob 543 and the corresponding distance the steering tube 539 is moved distally over the distal end portion 547 of the steering rod 535, or moved proximally uncovering the distal end portion 547 of the steering rod 535. Although the steering mechanism 538 is described herein with reference to clockwise rotation of the steering knob 543 causing the steering tube to move proximally, it should be understood that the steering mechanism can be configured (e.g., the threaded drive member 545 and drive nut 541) such that an opposite result is obtained.

As shown in the cross-sectional views of FIGS. 24 and 26, the flexible member 537 in this embodiment, includes a double layer of springs, and each of the two layers is coiled in an opposite direction from the other layer. Such a configuration enables the distal end portion of the medical device 520 to be maneuvered (steered or turned) in multiple directions and be returned to a linear configuration. For example, the distal end portion of the medical device 520 can be steered or turned in a first direction, and as the spring that is coiled in the first direction (referred to here as a first spring) is partially uncoiled (to allow for the turn) the second spring that is coiled in a direction opposite of the first spring applies torque in an opposite direction. This enables the flexible member 537 (i.e., the first spring) to move from a partially uncoiled configuration to a linear configuration. Thus, the two springs work together to allow the flexible member 537 to be moved back and forth between its curved configuration and its linear configuration.

In alternative embodiments, the steering tube can be configured to be actuated by other methods. For example, a medical device can be configured with a steering actuator that uses linear motion to cause the steering tube to move proximally and distally, rather than rotational motion (e.g., rotation of a steering knob). For example, a lever can be coupled to the steering tube that can be manually actuated by the user using linear motion. In other examples, a pull rod or a pulley mechanism can be used to move the steering tube. In another example, a fly-wheel mechanism can be coupled to the steering tube and used to move the steering tube proximally and distally. For example, the fly-wheel mechanism can have a lever arm that a user can turn or rotate to cause linear movement of the steering tube.

The medical device 520 can be used in a variety of different medical procedures as described above for other embodiments. In one example use, the expandable member 526 is collapsed and inserted through an access cannula to a desired location within an intervertebral disc in a similar manner as described above with reference to FIGS. 12 and 13. As the expandable member 526 emerges from a distal end of the access cannula (or from within a sheath coupled to the elongate body 522), it will assume its pre-set expanded configuration. The user can rotate the steering knob 543 to steer the distal end portion of the medical device 520 (e.g., move the flexible member 537 to a curved configuration) to a desired location within the intervertebral disc, as described above. The expandable member 526, coupled to a distal end of the flexible member 537, will in turn be moved to a desired location. As already described, the user can adjust the amount of curvature of the flexible member 537 to position the expandable member 526 at a desired location.

After the user has achieved the desired angle or position of the flexible member 537 and expandable member 526 within the intervertebral disc, the user can squeeze the lever 534 to actuate the actuation mechanism 534 and cause the elongate member 522, flexible member 537 and expandable member 526 to rotate. The arms 564 of the expandable member 526 will cut, tear, or disrupt tissue (e.g., nucleus material) within the intervertebral disc. As described above, the user can release the lever 534 to reset the actuation mechanism 524 and conversion mechanism 515, and then repeat the actuation of the medical device 520 as desired. The user can also optionally move the medical device 520 distally and proximally during the actuation.

The angle or curvature of the flexible member 537 can be adjusted as desired. For example, the user can rotate the steering knob 543 to move the flexible member 537 to a substantially linear configuration or a different angle of curvature to position the expandable member 526 at a different location within the intervertebral disc. The user can steer and reposition the medical device 520 to different locations within the intervertebral disc and then actuate the rotation of the expandable member 526 to disrupt nucleus material at various locations within the intervertebral disc. In some cases, it may be desired to disrupt the entire nucleus material within the intervertebral disc. Various regions within the intervertebral disc can be reached without removing and reinserting the medical device 520, which can help preserve the integrity of the annulus of the intervertebral disc. Thus, continuous disruption of nucleus material can be achieved by access through a single small opening in the annulus of the disc.

After the desired amount of disruption has been completed, the flexible member 537 can be moved to its linear or straight configuration and the expandable member 526 can be drawn proximally into the access cannula to remove the medical device 520 from the intervertebral disc. Irrigation and/or suction can then be applied to remove the disrupted nucleus material as described above via the access cannula or if the access cannula is removed, through the opening in the annulus of the intervertebral disc in which the cannula was placed. After the disrupted material has been removed from the intervertebral disc, a disc replacement procedure can then be performed. For example, a disc prosthesis can be implanted into the disc.

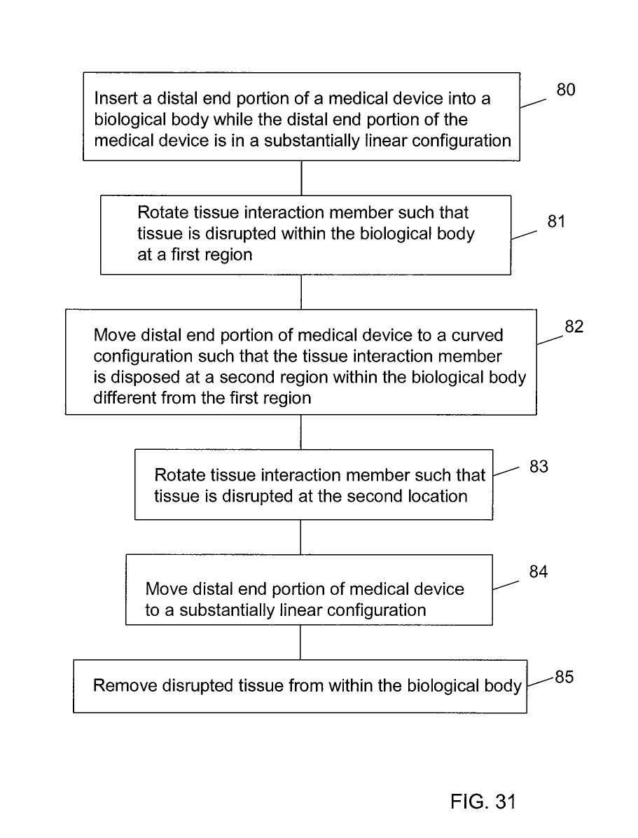

FIG. 29 shows a distal end portion of an embodiment of a medical device illustrating a tissue interaction member that is not expandable. In this embodiment, a tissue interaction member 626 is shown coupled to a flexible member 637 (similar to flexible member 537), which is coupled to an elongate member 622. The tissue interaction member 626 includes multiple teeth 687 that can be used to cut, tear, disrupt, and/or otherwise manipulate tissue when rotated within a biological body. Such a tissue interaction member 626 can be incorporated in any of the embodiments of a medical device described herein.

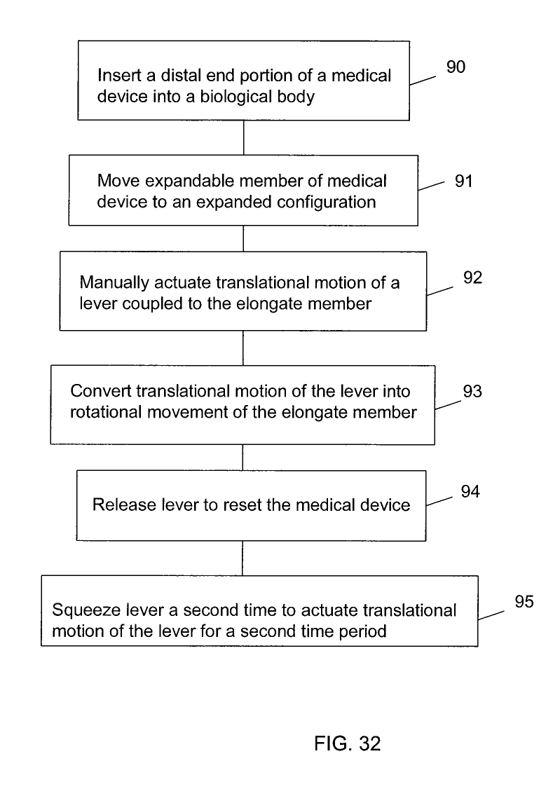

FIG. 30 is a flowchart illustrating an example of a method of disrupting tissue within a biological body. The method includes at 72, inserting a distal end portion of an elongate member of medical device (e.g., of a medical device 20, 120, 320, 420 and 520) into a biological body, such as a vertebra or an intervertebral disc. The medical device includes a tissue interaction member (e.g., an expandable member) disposed at a distal end of the elongate member. At 73, an actuation mechanism is manually actuated to produce translational motion of a drive element coupled to the elongate member. The actuation mechanism can include, for example, a lever coupled to a handle and to actuate the translational motion the lever is squeezed toward the handle. At 74, translational motion of the drive element is converted into rotational motion of the elongate member. As the elongate member rotates, tissue within the biological body can be disrupted by the tissue interaction member. In some embodiments, the rotational motion is in a single direction only. At 75, the medical device can be reset. For example, a lever of the actuator can be released to reset the medical device such that it can be actuated again. At 76, the actuation mechanism can be actuated a second time to actuate translational motion for a second time period.

FIG. 31 is a flowchart illustrating another method of disrupting tissue within a biological body. The method includes at 80, inserting a distal end portion of a medical device into a biological body while the distal end portion of the medical device is in a substantially linear configuration. The biological body can be for example, a vertebra or an intervertebral disc. The medical device includes a tissue interaction member disposed at a distal end of the medical device. The tissue interaction member is disposed at a first region within the biological body after being inserted.

At 81, the tissue interaction member is rotated such that tissue is disrupted within the biological body at the first region. In some embodiments, the rotation is in a single direction. At 82, the distal end portion of the medical device is moved to a curved configuration while disposed within the biological body such that the tissue interaction member is disposed at a second region within the biological body different from the first region. At 83, the tissue interaction member is rotated such that tissue is disrupted at the second location. At 84, the distal end portion of the medical device is moved to a substantially linear configuration. At 85, the disrupted tissue is removed from within the biological body.

FIG. 32 is a flowchart illustrating another example of a method of disrupting tissue within a biological body. The method includes at 90, inserting a distal end portion of a medical device (e.g., of a medical device 20, 120, 320, 420 and 520) into a biological body, such as a vertebra or an intervertebral disc. The medical device includes an elongate member and an expandable member disposed at a distal end of the elongate member. The expandable member is in a collapsed configuration when inserted into the biological body. At 91, the expandable member is moved to an expanded configuration. At 92, translational motion of a lever coupled to the elongate member is manually actuated. For example, a user can squeeze the lever toward a handle of the medical device. At 93, the translational motion of the lever is converted into rotational movement of the elongate member such that tissue within the biological body is disrupted by the expandable member when it is rotated. In some embodiments, the rotational motion is in a single direction only. The conversion of the translational motion can be performed during a time period associated with a distance the lever moves during the actuating. At 94, the lever is released to reset the medical device such that it can be actuated again. At 95, the lever is optionally squeezed a second time to actuate translational motion of the lever for a second time period.

Although the above described embodiments focus on a manually operated actuation mechanism, each of the embodiments of a medical device (e.g., 20, 120, 320, 420 and 520) can alternatively include features to allow for automated actuation of the device. For example, a battery or battery pack and motor can be included within the housing (e.g., within the handle) of the medical device and can be actuated between an on position or an off position with, for example, a button or switch accessible on an exterior of the housing. A user can then actuate the device to an on position to provide continuation rotation of the lead screw and elongate body until the device is moved to an off position. In some embodiments, a medical device can be configured to be powered with a power cord coupled to a power source (e.g., a wall outlet), rather than a battery pack. In such an embodiment, the device can be actuated with a button or switch as with a battery operated embodiment.

The medical device for any of the embodiments may be constructed with any suitable material used for such a medical device. The elongate member, the expandable member, and the steering rod for any embodiments can each be formed with nitinol, superelastic nitinol, or other shape-memory material. The various components of the medical device (20, 120, 320, 420, 520) can each be formed with various biocompatible metal materials, such as stainless steel, titanium, titanium alloy, surgical steel, metal alloys, or suitable biocompatible plastic materials, such as various polymers, polyetheretherketone (PEEK), carbon fiber, ultra-high molecular weight (UHMW) polyethylene, etc., or various elastic materials, flexible materials, various rubber materials, or combinations of various materials thereof. The flexible expandable member can be formed with various flexible or expandable materials such as plastics (e.g., various polymers) and/or rubber materials having flexible or pliable characteristics.

While various embodiments of the invention have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

For example, although various embodiments have been described as having particular features and/or combinations of components, other embodiments are possible having any combination or sub-combination of any features and/or components from any of embodiments described herein. For example, although the steering mechanism was described with reference to medical device 520, a steering mechanism can be incorporated in any of the embodiments of a medical device. In addition, a manually translated sheath, such as a sheath 136 shown in FIG. 12, can be included in any embodiment of a medical device, or a translating sheath coupled to the actuation mechanism as described with reference to medical device 320 can be included in any embodiment.

Further, the various components of a medical device as described herein can have a variety of different shapes and or size not specifically illustrated. For example, the expandable members can include various quantities of arms, and/or can be a variety of different shapes or sizes. The elongate member can be a various lengths and have various cross-sections. The elongate member can have a lumen or can be solid.

Also, the handle, actuation mechanism, conversion mechanism, and/or steering mechanism can be used to actuate other types of tissue interaction members not specifically described. For example, although the medical devices described herein included an elongate member having an expandable member disposed at a distal end thereof, other types of tissue interaction members can alternatively be incorporated in a medical device as described herein. For example, other types and configurations of scraping, cutting, curetting, disrupting, or debulking tools can be used. In addition, the use of a sheath, such as a sheath 136, may not be needed depending on the particular configuration of the tissue interaction member. For example, a sheath may not be needed to collapse a tissue interaction member that does not have an expanded configuration as described herein.

Although the use of a medical device was described with a specific example of use within a vertebra and intervertebral disc, it should be understood that the medical device and methods described herein can be used in other areas of a patient. For example, the medical device can be used in other areas within a spine, as well as other bone or soft tissue areas within a patient's body.

What is claimed is:

1. A method, comprising:
inserting a distal end portion of an elongate member into a biological body;
after the inserting, manually actuating an actuation mechanism to produce translational motion of a drive element; and
converting the translational motion into rotational movement of the distal end portion of the elongate member.

2. The method of claim 1, wherein the actuation mechanism includes a handle coupled to a lever, the actuating includes a user squeezing the lever toward the handle.

3. The method of claim 1, wherein the drive element includes a first threaded element, the converting the translational motion includes preventing the first threaded element from rotating during the translational motion and engaging the first threaded element with a second threaded element during the translational motion,
the second threaded element being coupled to a proximal end portion of the elongate member.

4. The method of claim 1, wherein the actuating includes a user squeezing the lever toward a handle coupled to the lever, the method further comprising:
releasing the lever; and
after the releasing, squeezing the lever to actuate translational motion of the drive element.

5. The method of claim 1, wherein the actuation mechanism includes a handle coupled to a lever, the actuating includes a user squeezing the lever toward the handle,
the converting is performed when the lever is squeezed toward the handle.

6. The method of claim 1, wherein the distal end portion of an elongate member is in a collapsed configuration during the inserting, the method further comprising:
after the inserting, moving the distal end portion of the elongate member to an expanded configuration.

7. The method of claim 1, wherein the inserting includes inserting the distal end portion of the elongate member into an interior of a vertebra, the method further comprising:
disrupting cancellous bone within the vertebra when the distal end portion of the elongate body is rotated.

8. The method of claim 1, wherein the rotational movement is in a single direction.

9. An apparatus, comprising:
an elongate member having a tissue interaction member at a distal end of the elongate member, the tissue interaction member configured to be inserted within a biological body;
a conversion mechanism coupled to the elongate member, the conversion mechanism including a drive element; and
an actuator coupled to the conversion mechanism and configured to cause translational motion of the drive element,
the conversion mechanism configured to convert the translational motion of the drive element into rotational motion of the elongate member.

10. The apparatus of claim 9, wherein the tissue interaction member includes a plurality of arms configured to disrupt tissue within the biological body.

11. The apparatus of claim 9, wherein the drive element includes a threaded member, the elongate member includes a lead screw portion threadedly mated with the threaded member, and
the actuator includes a retention member configured to prevent rotation of the threaded member during the translational motion of the drive element in at least a first direction.

12. The apparatus of claim 9, wherein the actuator includes a rotation-limiting mechanism coupled to the elongate member, the rotation-limiting mechanism is configured to allow rotation of the elongate member only in a single direction.

13. The apparatus of claim 9, further comprising:
a sheath coupled to the actuator, the elongate member disposable within a lumen of the sheath, the actuator configured to move the sheath relative to the elongate member.

14. The apparatus of claim 9, further comprising:
a sheath coupled to the actuator, the elongate member disposable within a lumen of the sheath, the tissue interaction member being in a collapsed configuration when disposed within a lumen of the sheath.

15. The apparatus of claim 9, wherein the conversion mechanism is configured to rotate the elongate member in a first direction and prevent the elongate member from rotating in a second direction opposite the first direction.

16. An apparatus, comprising:
an actuator;
a conversion mechanism coupled to the actuator;
an elongate member coupled to the conversion mechanism, the conversion mechanism configured to convert translational motion of the actuator into rotational motion of the elongate member; and
a tissue interaction member disposed at a distal end of the elongate member, the tissue interaction member configured to be inserted within a biological body, the conversion mechanism configured to rotate the elongate member when the actuator is actuated by a user,
the tissue interaction member configured to disrupt tissue in the biological body when the elongate member is rotated.

17. The apparatus of claim 16, wherein tissue interaction member is an expandable member including a plurality of arms configured to disrupt tissue within the biological body.

18. The apparatus of claim 16, wherein the conversion mechanism includes a lead screw coupled to the elongate member.

19. The apparatus of claim 16, further comprising:
a sheath coupled to the actuator, the elongate member disposable within a lumen of the sheath.

20. The apparatus of claim 16, further comprising:
a sheath coupled to the actuator, the tissue interaction member being an expandable member disposable within a lumen of the sheath, the expandable member being in a collapsed configuration when disposed within a lumen of the sheath.

21. The apparatus of claim 16, wherein the actuator includes a lever, the conversion mechanism is configured to transform translational movement of the lever into rotational movement of the elongate member.

22. An apparatus, comprising:
a sheath;
an elongate member movably disposed within a lumen of the sheath;
an expandable member disposed at a distal end of the elongate member;
a conversion mechanism configured to rotate the elongate member; and
an actuator coupled to the sheath, the actuator configured to translate the sheath between a first position in which the expandable member is in a collapsed configuration disposed within a lumen of the sheath and a second position in which the expandable member is in an expanded configuration disposed outside of the lumen of the sheath,
the expandable member configured to disrupt tissue within a biological body when the expandable member is in the expanded configuration and disposed outside the lumen of the sheath and within the biological body.

23. The apparatus of claim 22, wherein the expandable member is monolithically formed with the elongate member.

24. The apparatus of claim 22, wherein the expandable member includes a plurality of arms.

25. The apparatus of claim 22, wherein the elongate member defines a lumen, a proximal end of the elongate member configured to be coupled to a suction source to remove disrupted tissue from the biological body and through the lumen of the elongate member.

26. The apparatus of claim 22, wherein the expandable member includes a plurality of arms that define an interior region, the plurality of arms being configured to capture disrupted tissue within the interior region when the expandable member is moved from an expanded configuration to a collapsed configuration.
